(19) United States
(12) Patent Application Publication
(10) Pub. No.: US 2023/0233573 A1
(43) Pub. Date: Jul. 27, 2023

(54) TREATING INFLUENZA USING SUBSTITUTED POLYCYCLIC PYRIDONE DERIVATIVES AND PRODRUGS THEREOF

(71) Applicant: SHIONOGI & CO., LTD., Osaka (JP)

(72) Inventors: Takao SHISHIDO, Osaka (JP); Keita FUKAO, Osaka (JP); Takeshi NOSHI, Osaka (JP); Yoshinori ANDO, Osaka (JP); Takahiro NODA, Osaka (JP)

(21) Appl. No.: 18/192,491

(22) Filed: Mar. 29, 2023

Related U.S. Application Data
(63) Continuation of application No. 17/204,246, filed on Mar. 17, 2021.
Application No. 17/204,246 is a continuation of application No. PCT/IB2018/057192, filed on Sep. 18, 2018.

(51) Int. Cl. A61K 31/5383; A61P 31/16; A61K 9/00; A61K 9/06; A61K 9/28; A61K 9/48; A61K 9/70; A61K 31/245; A61K 45/06

(52) U.S. Cl. CPC A61K 31/5383; A61P 31/16; A61K 9/0019; A61K 9/0053; A61K 9/0056; A61K 9/007; A61K 9/0095; A61K 9/06; A61K 9/282; A61K 9/2826; A61K 9/4858; A61K 9/7007; A61K 31/245; A61K 45/06

(57) ABSTRACT

A method for treating influenza is described. The disclosed method generally involves administering an effective amount of a compound, for example baloxavir marboxil, to a subject having influenza, where the compound is administered initially at least about 48 hours after an onset of influenza. Generally, the effective amount is sufficient to alleviate a symptom of influenza in the subject as compared to a symptom that the subject has when the compound is first administered to the subject.

### FIELD

The present disclosure relates generally to treating influenza using a substituted polycyclic pyridone derivative having cap-dependent endonuclease inhibitory activity, a prodrug thereof, and a pharmaceutical composition including thereof.

### BACKGROUND

Influenza is an acute respiratory infectious disease caused by infection with an influenza virus. In Japan, millions of influenza-like patients are reported every winter, and influenza is accompanied with high morbidity and mortality. Influenza is a particularly important disease in a high risk population such as babies and the elderly, a complication rate with pneumonia is high in the elderly, and influenza has been a cause of death in many elderlies.

As anti-influenza drugs, Symmetrel (trade name: Amantazine) and Flumadine (trade name: Rimantadine) which inhibit the denucleation process of a virus, and Oseltamivir (trade name: Tamiflu) and Zanamivir (trade name: Relenza) which are neuraminidase inhibitors suppressing virus budding and release from a cell are known. However, there are problems of appearances of resistant strains and side effects, and a worldwide epidemic of a new-type influenza virus having high pathogenicity and mortality is feared, so development of an anti-influenza drug having a novel mechanism has been desired.

Since a cap-dependent endonuclease, which is an influenza virus-derived enzyme, is essential for virus proliferation, and has the virus-specific enzymatic activity which is not possessed by a host, it is believed that the endonuclease is suitable for a target of an anti-influenza drug. The cap-dependent endonuclease of an influenza virus has a host mRNA precursor as a substrate, and has the endonuclease activity of producing a fragment of 9 to 13 bases including a cap structure (not including the number of bases of the cap structure). This fragment functions as a primer of a virus RNA polymerase, and is used in synthesizing mRNA encoding a virus protein. That is, it is believed that a substance which inhibits the cap-dependent endonuclease inhibits synthesis of a virus protein by inhibiting synthesis of virus mRNA and, as a result, inhibits virus proliferation.

Generally known compounds are provided as follows. As a substance which inhibits the cap-dependent endonuclease, flutimide (Patent Document 1 and Non-Patent Documents 1 and 2), 4-substituted 2,4-dioxobutanoic acid (Patent Document 2 and Non-Patent Documents 3 and 4), the compounds described in Patent Documents 3 to 12 and the like have been reported, but they have not yet led to clinical use as anti-influenza drugs. Patent Documents 9 and 12 describe compounds having similar structures to substituted polycyclic pyridone derivatives. Also, Patent Documents 13 to 15 describe compounds having a similar structure to substituted polycyclic pyridone derivatives. These documents do not describe cap-dependent endonuclease. In addition, Patent Document 16 and 17 further describe compounds having similar structures to substituted polycyclic pyridone derivatives.
Patent Document 1: GB2280435
Patent Document 2: U.S. Pat. No. 5,475,109
Patent Document 3: US20130090300
Patent Document 4: WO2013/057251
Patent Document 5: WO2013/174930
Patent Document 6: WO2014/023691
Patent Document 7: WO2014/043252
Patent Document 8: WO2014/074926
Patent Document 9: WO2014/108406
Patent Document 10: WO2014/108407
Patent Document 11: WO2014/108408
Patent Document 12: WO2015/038655
Patent Document 13: WO2005/016927
Patent Document 14: WO2006/066414
Patent Document 15: WO2007/049675
Patent Document 16: WO2010/147068
Patent Document 17: WO2012/039414
Non-Patent Document 1: Tetrahedron Lett 1995, 36(12), 2005
Non-Patent Document 2: Tetrahedron Lett 1995, 36(12), 2009
Non-Patent Document 3: Antimicrobial Agents And Chemotherapy, December 1994, p. 2827-2837
Non-Patent Document 4: Antimicrobial Agents And Chemotherapy, May 1996, p. 1304-1307

Currently, neuraminidase (NA) inhibitors are the most widely used class of anti-influenza drug that inhibit influenza A and B viruses. Previous clinical studies indicated that administration with NA inhibitors is required to be initiated within 48 hours of the onset of symptoms. To date, however, there is a possibility that the treatment may not be initiated within 48 hours due to delayed diagnosis of pathogens. In addition, there are concerns of the emergence of influenza viruses resistant to NA inhibitors. Currently, there is no dosage allowed or approved by the health authorities of a pharmaceutical compositions including NA inhibitors for initiating the treatment with the pharmaceutical compositions at or after 48 hours of the onset of symptoms of influenza. There further are no such dosage instructions on a package insert or in a package thereof. Thus, a more effective anti-influenza drug and treatment that can extend the therapeutic window, for example those effective when initially applied to patients at or after 48 hours of the onset of symptoms, have been needed.

### SUMMARY

A method for treating influenza is described. The disclosed method generally involves administering an effective amount of a compound to a subject having influenza, where the compound is administered initially at at least about 48 hours after an onset of influenza.

In one example, the compound has one of the following formulae:

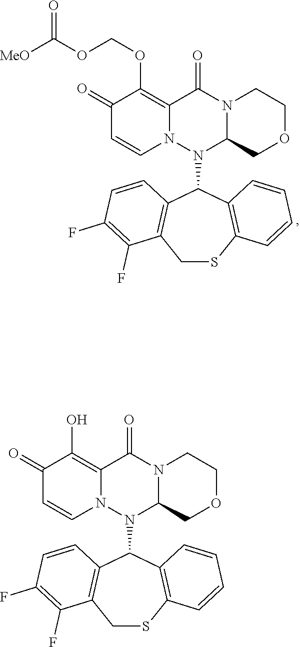 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or a pharmaceutically acceptable salt thereof.

In one example, the number of times the compound is administered is not particularly limited. In another example, the compound can be administered only once. In another example, the compound can be administered only two times. In another example, the compound can be administered only three times.

In one example, the onset of influenza in the subject is when the subject has a virus titer sufficient to cause a symptom of influenza to be exhibited in the subject. In one example, the onset of influenza in the subject is when the subject initially exhibits an increase in a body temperature from a normal temperature of the subject. In another example, the onset of influenza in the subject is when the subject initially exhibits an increase in a body temperature of at least 1° C. from a normal temperature of the subject. In one example, the onset of influenza in the subject is when the subject initially develops a systemic symptom or a respiratory symptom. In one example, a systemic symptom includes one or more of headache, feverishness, chills, muscular pain, joint pain, and fatigue. In one example, a respiratory symptom includes one or more of coughing, sore throat, and nasal congestion.

In one example, the effective amount is sufficient to alleviate a symptom of the disease in the subject as compared to a symptom that the subject has when the compound is first administered to the subject. In one example, the effective amount is sufficient to reduce an amount of the virus in the subject as compared to an amount of the virus that the subject has when the compound is first administered to the subject.

In one example, the effective amount of the compound is in a range from at or about 0.1 to at or about 3000 mg. In another example, the effective amount of the compound is in a range from about at or 0.1 to at or about 240 mg. In another example, the effective amount of the compound is in a range from about at or 5 to at or about 80 mg. In yet another example, the effective amount of the compound is in a range from at or about 40 to at or about 80 mg. In yet another example, the effective amount is in a range from at or about 10 to at or about 80 mg per dose.

In one example, the compound is administered at or before about 120 hours after the onset of the disease in the subject. In one example, the compound is administered at or before about 96 hours after the onset of the disease in the subject.

### BRIEF DESCRIPTION OF THE DRAWINGS

FIG. 1 is a graph showing the experimental results of measuring the plasma concentration of compound 111-2, after oral administration of prodrug compound 11-6 (BXM), whose parent compound is compound 111-2, to rat under non-fasting conditions.

FIG. 2 is a table showing the experimental results of measuring the plasma concentration of compound 11-6 (BXM), after oral administration of prodrug compound 11-6, whose parent compound is compound 111-2, to rat under non-fasting conditions.

FIGS. 3A-D are graphs of experimental results showing the therapeutic efficacy of delayed administration of compound 11-6 (BXM) against lethal influenza A virus infection in mice. Ten mice per group (except for the uninfected mice (n=5)) infected with A/PR/8/34 (1.38×10³ TCID₅₀) were treated orally with BXM (baloxavir marboxil), OSP (oseltamivir phosphate), or vehicle twice daily for 5 days from (FIG. 3A) 24, (FIG. 3B) 48, (FIG. 3C) 72, or (FIG. 3D) 96 hours post infection, respectively. Mice were monitored daily for survival and body weight through 28 days post infection. Significant differences in survival time were observed in groups treated with BXM from 24, 48, 72, and 96 hours post infection in comparison with vehicle-treated group (**, P<0.01; ***, P<0.001). The survival time in groups that treated with OSP from 24 and 48 hour post infection was significantly prolonged compared to that in vehicle-treated group (**, P<0.01; ***, P<0.001). The survival time of the group that received BXM starting at 72 and 96 hours post infection was significantly prolonged compared to that of the groups treated with OSP at a dose of 5 mg/kg (†, P<0.001; ††, P<0.0001).

FIGS. 4A-H are graphs of experimental results Effects of delayed administration of compound 11-6 on body weight change following in influenza virus infection. Mice infected with A/PR/8/34 (1.38×10³ TCID₅₀) were treated as described in the legend respectively in each FIGS. 4A-H, orally with BXM, OSP, or vehicle twice daily for 5 days from (FIG. 4A and FIG. 4E) 24, (FIG. 4B and FIG. 4F) 48, (FIG. 4C and FIG. 4G) 72, or (FIG. 4D and FIG. 4H) 96 hours post infection, respectively and monitored daily for body weight up to 28 days post infection. Significant differences in body weight were observed in groups treated with BXM and OSP in comparison with vehicle-treated group on indicated days (*, P<0.05; **, P<0.01; ***, P<0.001). The groups treated with BXM from 24, 48, 72 or 96 hour post infection showed significantly less body weight loss than did the OSP-treated group on indicated days (†, P<0.05; ††, P<0.01, †††, P<0.0001).

FIG. 5 is a graph of experimental results showing the inhibitory effects of delayed administration of compound 11-6 on virus replication in mice. Mice infected with A/PR/8/34 (1.38×10³ TCID₅₀) were treated as described in the legend for FIG. 5, orally with BXM, OSP, or vehicle twice daily for 5 days from 72 hours post infection and were euthanized at indicated days. The virus titers in lungs were measured by the TCID₅₀ method. Each point represents the mean±SD of 6 to 8 mice except points that indicated virus titer on days 8 and 10 in mice treated with OSP (N=1), in which only one mouse survived. No virus was detected in mice treated BXM and OSP on days 8 and 10. The limit of detection (1.50 logo TCID₅₀/ml) is indicated by a dotted line. Significant differences in virus titers were observed in BXM and OSP-treated groups in comparison with the vehicle-treated group on the days 4 and 6 and day 4 post infection, respectively (**, P<0.01; ***, P<0.001). Significant differences in virus titers were also observed between BXM and OSP-treated groups on days 4 and 6 post infection (†††, P<0,0001).

### DETAILED DESCRIPTION

A method for treating a disease caused by influenza is described. The disclosed method generally involves administering an effective amount of a compound to a subject having influenza, where the compound is administered initially at least about 48 hours after an onset of influenza in the subject.

Generally, the compound that can be used in the disclosed is described as follows.

(1) A compound represented by the following formula:

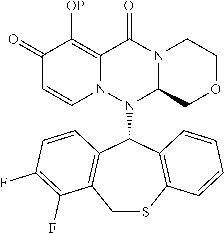

wherein P is hydrogen or a group to form a prodrug, or its pharmaceutically acceptable salt.
(2) The compound according to (1), or its pharmaceutically acceptable salt, wherein the group to form a prodrug is a group selected from the following formula:

—C(═O)—P^{R0}, a)

—C(═O)—O—P^{R2}, g)

—C(═O)—O-L-O—P^{R2}, i)

—C(P^{R3})₂—O—C(═O)—P^{R4}, l)

—C(P^{R3})₂—O—C(═O)—O—P^{R4}, and i)

—C(P^{R3})₂—O—C(═O)—O-L-O—P^{R4} i)

wherein L is straight or branched lower alkylene;
P^{R0} is alkyl;
P^{R2} is alkyl;
P^{R3} is each independently hydrogen; and
P^{R4} is alkyl.

In one example, the compound that can be used in the disclosed method has a formula:

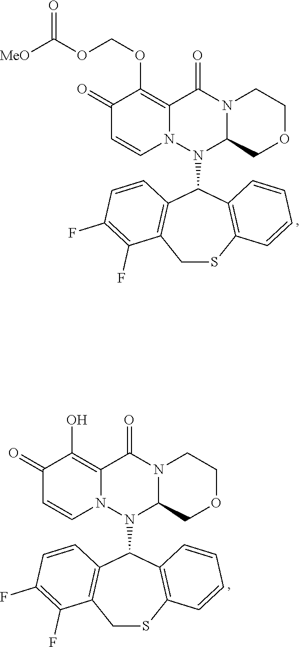 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or its pharmaceutically acceptable salt.

The meaning of each term used in the present description is explained below. Each term is used in a unified sense, and is used in the same sense when used alone, or when used in combination of other term.

The term of “consisting of” means having only components.

The term of “comprising” means not restricting with components and not excluding undescribed factors.

“Prodrug” in the present description refers to a compound represented by formula (II) in the following reaction formula:

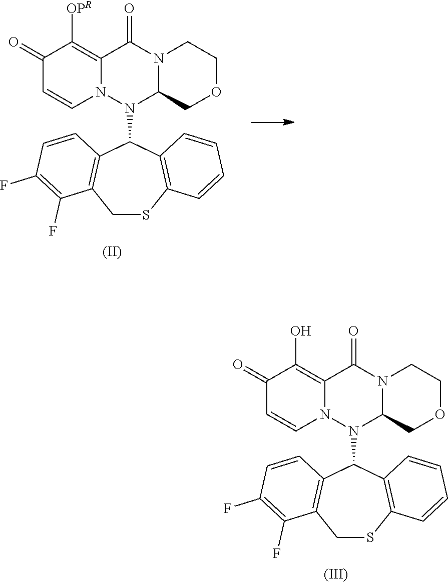

wherein each symbol is same as the above,
or its pharmaceutically acceptable salt, and means a compound showing cap-dependent endonuclease (CEN) inhibitory activity and/or CPE inhibitory effect by being converted into a compound represented by formula (III) by a decomposition reaction caused by drug-metabolizing enzymes, hydrolases, gastric acids, enterobacteria, etc. under physiological conditions in vivo.

The prodrug means a compound in which bioavailability and/or AUC (area under the blood concentration curve) in in vivo administration is improved more than those of the compound represented by formula (III).

Therefore, the prodrug is efficiently absorbed into the body in the stomach and/or intestines after in vivo administration (for example, oral administration), then converted into the compound represented by formula (III). Thus, the prodrug shows an effect of treating and/or preventing influenza higher than the compound represented by formula (III).

“Group to form a prodrug” in the present description refers to a “P^{R}” group in the formula (II), in the following reaction formula:

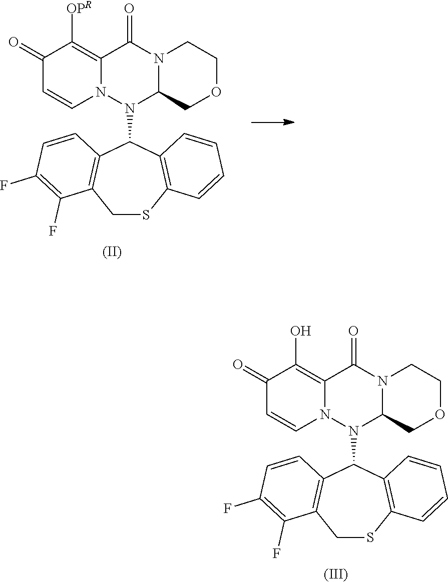

wherein each symbol is same as the above,
and —OP^{R} group is converted into —OH group in the formula (III) by a decomposition reaction caused by drug-metabolizing enzymes, hydrolases, gastric acids, enterobacteria, etc. under physiological conditions in vivo.

The “group to form a prodrug” means a group that improves bioavailability and/or AUC (area under the blood concentration curve) of the compound represented by formula (III) by being added to the compound represented by formula (III).

Examples of the group P^{R} to form a prodrug include the groups described in Prog. Med. 5: 2157-2161 (1985) and Supplied by The British Library—“The world's Knowledge”.

The “group to form a prodrug” in —OP^{R} group in the formula (II) may be a group converted into —OH group in vivo, and examples include a group selected from the following formulae.

—C(═O)—P^{R0}, a)

—C(═O)—O—P^{R2}, g)

—C(═O)—O-L-O—P^{R2}, i)

—C(P^{R2})₂—O—C(═O)—P^{R4}, l)

—C(P^{R3})₂—O—C(═O)—O—P^{R4}, and m)

—C(P^{R3})₂—O—C(═O)—O-L-O—P^{R4} o)

wherein L is straight or branched lower alkylene;
P^{R0} is alkyl;
P^{R2} is alkyl;
P^{R3} is each independently hydrogen; and
P^{R4} is alkyl.

“Converted into a prodrug” in the present description means that, as shown in the following reaction formula:

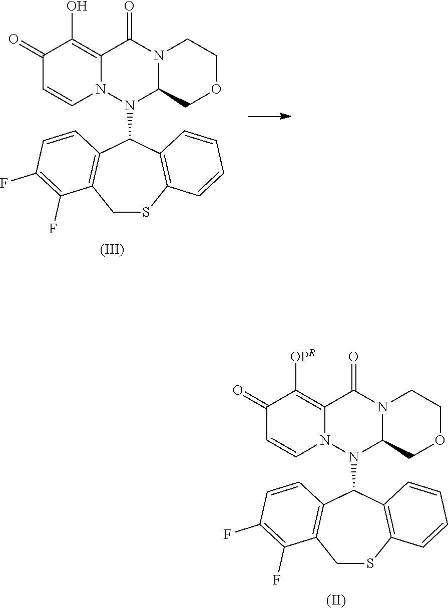

wherein each symbol is same as the above,
a hydroxy group in the formula (III) or its pharmaceutically acceptable salt is converted into —OP^{R} group.

“Parent compound” in the present description means a compound to be a source before synthesizing the “prodrug” and/or a compound released from the “prodrug” by the reaction by enzymes, a gastric acid, and the like under physiological conditions in vivo, and specifically means a compound shown by the formula (III), or pharmaceutically acceptable salt thereof or a solvate thereof.

The term “alkyl” includes a C1 to C15, alternatively a C1 to C10, alternatively a C1 to C6, alternatively a C1 to C4, linear or branched hydrocarbon group. Examples include methyl, ethyl, n-propyl, isopropyl, n-butyl, isobutyl, sec-butyl, tert-butyl, n-pentyl, isopentyl, neopentyl, n-hexyl, isohexyl, n-heptyl, isoheptyl, n-octyl, isooctyl, n-nonyl, n-decyl and the like.

One embodiment of “alkyl” is methyl, ethyl, n-propyl, isopropyl, n-butyl, isobutyl, sec-butyl, tert-butyl or n-pentyl. One embodiment is methyl, ethyl, n-propyl, isopropyl or tert-butyl.

The term “alkylene” includes a C1 to C15, alternately a C1 to C10, alternately a C1 to C6 and alternately a C1 to C4 liner or branched bivalent hydrocarbon group. Examples include methylene, ethylene, trimethylene, propylene, tetramethylene, pentamethylene, hexamethylene and the like.

One or more hydrogen, carbon and/or other atoms in the compounds used in the present invention may be replaced with isotopes of hydrogen, carbon and/or other atoms respectively. Examples of isotopes include hydrogen, carbon, nitrogen, oxygen, phosphorus, sulfur, fluorine, iodine and chlorine, such as ²H, ³H, ¹¹C, ¹³C, ¹⁴C, ¹⁵N, ¹⁸O, ¹⁷O, ³¹P, ³²P, ³⁵S, ¹⁸F, ¹²³I and ³⁶Cl respectively. The compounds used in the present invention include compounds replaced with these isotopes. The compounds replaced with the above isotopes are useful as medicines and include all of radiolabeled compounds of the compound used in the present invention. A “method of radiolabeling” in the manufacture of the “radiolabeled compounds” is encompassed by the present invention, and the “radiolabeled compounds” are useful for studies on metabolized drug pharmacokinetics, studies on binding assay and/or diagnostic tools.

A radiolabeled compound used in the present invention can be prepared using well-known methods in this field of the invention. For example, a tritium-labeled compound used in the present invention can be prepared by introducing a tritium to a certain compound used in the present invention, through a catalytic dehalogenation reaction using a tritium. This method comprises reacting with an appropriately-halogenated precursor of the compound used in the present invention with tritium gas in the presence of an appropriate catalyst, such as Pd/C, and in the presence or absent of a base. The other appropriate method of preparing a tritium-labeled compound can be referred to “Isotopes in the Physical and Biomedical Sciences, Vol. 1, Labeled Compounds (Part A), Chapter 6 (1987)”. A ¹⁴C-labeled compound can be prepared by using a raw material having ¹⁴C.

The pharmaceutically acceptable salts of the compounds used in the present invention include, for example, salts with alkaline metal (e.g., lithium, sodium, potassium or the like), alkaline earth metal (e.g., calcium, barium or the like), magnesium, transition metal (e.g., zinc, iron or the like), ammonia, organic bases (e.g., trimethylamine, triethylamine, dicyclohexylamine, ethanolamine, diethanolamine, triethanolamine, meglumine, ethylenediamine, pyridine, picoline, quinoline or the like) or amino acids, or salts with inorganic acids (e.g., hydrochloric acid, sulfuric acid, nitric acid, carbonic acid, hydrobromic acid, phosphoric acid, hydroiodic acid or the like) or organic acids (e.g., formic acid, acetic acid, propionic acid, trifluoroacetic acid, citric acid, lactic acid, tartaric acid, oxalic acid, maleic acid, fumaric acid, mandelic acid, glutaric acid, malic acid, benzoic acid, phthalic acid, ascorbic acid, benzenesulfonic acid, p-toluenesulfonic acid, methanesulfonic acid, ethanesulfonic acid or the like). Especially, salts with hydrochloric acid, sulfuric acid, phosphoric acid, tartaric acid, methanesulfonic acid and the like are included. These salts can be formed by the usual methods.

The compounds used in the present invention or its pharmaceutically acceptable salts may form solvates (e.g., hydrates or the like) and/or crystal polymorphs. The present invention encompasses those various solvates and crystal polymorphs. “Solvates” may be those wherein any numbers of solvent molecules (e.g., water molecules or the like) are coordinated with the compounds used in the present invention. When the compounds used in the present invention or its pharmaceutically acceptable salts are allowed to stand in the atmosphere, the compounds may absorb water, resulting in attachment of adsorbed water or formation of hydrates. Recrystallization of the compounds used in the present invention or its pharmaceutically acceptable salts may produce crystal polymorphs.

The group to form a prodrug is converted into OH group by action of drug-metabolizing enzymes, hydrolases, gastric acids, and/or enterobacteria, after in vivo administration (for example, oral administration).

Examples of one embodiment of the group to form a prodrug include a group selected from the following formulae.

—C(═O)—P^{R0}, a)

—C(═O)—O—P^{R2}, g)

—C(═O)—O-L-O—P^{R2}, i)

—C(P^{R3})₂—O—C(═O)—P^{R4}, l)

—C(P^{R3})₂—O—C(═O)—O—P^{R4}, and m)

—C(P^{R3})₂-O—C(═O)—O-L-O—P^{R4} o)

wherein L is straight or branched lower alkylene;
P^{R0} is alkyl;
P^{R2} is alkyl;
P^{R3} is each independently hydrogen; and
P^{R4} is alkyl.

Examples of an embodiment of a particularly preferable substituent of the group to form a prodrug include following groups.

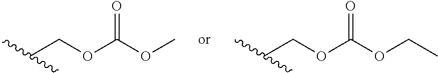

Other compounds that may be used are described in PCT application PCT/JP2016/063139 and publication WO 2016/175224A1, all disclosures in which are herein incorporated by reference.

A general method for producing the compound used in the present invention will be exemplified below. As to the extraction and purification, treatment which is performed in a normal experiment of organic chemistry may be conducted.

Synthesis of the compound used in the present invention can be carried out referring to the procedures known in the art.

As a raw material compound, commercially available compounds, compounds described in the present description, compounds described in the references cited in the present description, and other known compounds can be utilized.

When one wants to obtain a salt of the compound used in the present invention, in the case where the compound used in the present invention is obtained in a form of a salt, it may be purified as it is and, in the case where the compound used in the present invention is obtained in a free form, a salt may be formed by a normal method by dissolving or suspending the compound in a suitable organic solvent, and adding an acid or a base.

In addition, the compound used in the present invention and a pharmaceutically acceptable salt thereof are present in a form of adducts with water or various solvents (hydrate or solvate) in some cases, and these adducts are included in the present invention.

In a general synthesis method as well as Reference examples, Examples, and Intermediate Synthesis Examples, the meaning of each abbreviation is as follows.

### DMA: N,N-dimethylacetamide

OBn: benzyloxy
THF: tetrahydrofuran
T3P: propyl phoshonic anhydride

The up and down of the “wedge” and “broken line wedge” indicates the absolute configuration.

### (Preparation 1)

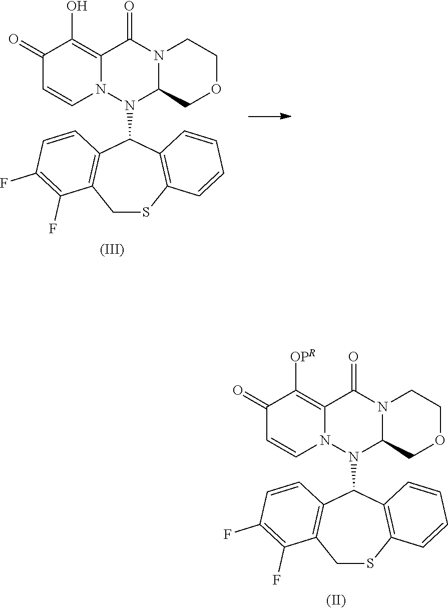

Compound (II) can be obtained by the general method including converting a hydroxyl group of Compound (III) into an ester group or ether group.

For example, the method described in Protective Groups in Organic Synthesis, Theodora W Green (John Wiley & Sons), Prog. Med. 5: 2157-2161 (1985), and Supplied by The British Library—“The world's Knowledge”, etc. can be utilized. These references are herein incorporated by reference.

The parent compound used in the present invention has cap-dependent endonuclease inhibitory activity and the parent compound and its prodrugs are useful as a therapeutic or preventive agent for influenza.

The compound used in the present invention not only has cap-dependent endonuclease inhibitory activity but also is useful as a medicine and has any or all of the following excellent characteristics:

a) The compound is a weak inhibitor of CYP enzymes (e.g., CYP1A2, CYP2C9, CYP2C19, CYP2D6, CYP3A4 and the like).

b) The compound demonstrates good pharmacokinetics, such as a high bioavailability, moderate clearance and the like.

c) The compound has a high metabolic stability.

d) The compound has no irreversible inhibitory action against CYP enzymes (e.g., CYP3A4) when the concentration is within the range described in the present description as the measurement conditions.

e) The compound has no mutagenicity.

f) The compound is associated with a low cardiovascular risk.

g) The compound has a high solubility.

h) The compound has no phototoxicity.

In general, for the purpose of treating the above-mentioned diseases in humans, the compounds used in the present invention may be administered orally as a powder, a granule, tablets, capsules, pills, a liquid and the like or parenterally as an injection, suppositories, a percutaneous drug, an inhalant and the like. The effective doses of the present compounds may be mixed with excipients suitable for the dosage form, such as fillers, binders, humectants, disintegrators, and lubricants, as appropriate, to form pharmaceutical preparations. For preparing an injection, sterilization is performed with a suitable carrier.

In general, the pharmaceutical compositions used in the present invention can be administered either orally or parenterally. For oral administration, commonly used dosage forms, such as tablets, granule, powder, and capsules, may be prepared according to conventional methods. For parenteral administration, any commonly used dosage form, such as an injection, may be suitably used. The compounds according to the present invention can be suitably used as oral preparations because of their high oral absorbability.

The effective doses of the compounds used in the present invention can be mixed with various pharmaceutical excipients suitable for the dosage form, such as fillers, binders, disintegrators, and lubricants, as appropriate, to form pharmaceutical compositions.

Generally, the dose depends on the condition of the disease, administration route, or age or weight of the patient. The usual oral dose for adults is 0.1 to 100 mg/kg per day, alternately 1 to 20 mg/kg per day.

In general, the compound used in the present invention can be used in combination with other drugs or the like (hereinafter referred to as combination drugs) to increase the activity of the compound, reduce the dose of the compound, or the like. In the case of treating influenza, the compound can be used combined with or in a coupled formulation with neuraminidase inhibitor (e.g., Oseltamivir, Zanamivir, Peramivir, Inabiru and the like); RNA-dependent RNA polymerase inhibitor (e.g., Favipiravir); M2 protein inhibitor (e.g., Amantadine); PB2 Cap binding inhibitor (e.g., VX-787); anti-HA antibody (e.g., MHAA4549A); Immune agonists (e.g., Nitazoxanide) are also possible. In this case, the timing of administration for a compound used in the present invention and the combination drug is not limited. They can be administered to the subjects to be treated, at a time or at different times. Furthermore, a compound used in the present invention and the combination drug can be administered as two or more formulations independently comprising each active ingredient or a single formulation comprising each active ingredient.

The dose for combination drugs may be appropriately selected in reference to the clinical dose. The compounding ratio of the compounds used in the present invention and co-administered drugs may be appropriately selected depending on the subject to be treated, administration route, disease to be treated, symptoms, combination of the drugs and the like. For administration in humans, for example, 1 part by weight of the compounds used in the present invention may be used in combination with 0.01 to 100 parts by weight of co-administered drugs.

The present invention will be explained in more detail below by way of Examples, Reference examples, Intermediate Synthesis Examples, as well as Test Examples of the present invention, but the present invention is not limited to them.

The NMR analysis obtained in each reference example and example was carried out in 300 MHz, and was measured using DMSO-d₆, CDCl₃.

The term RT represents a retention time at LC/MS: liquid chromatography/mass spectrometry, and was measured under the following conditions.

### (Measurement Conditions)

(1) Column: ACQUITY UPLC (Registered trademark) BEH C18 (1.7 μm i.d.2.1×50 mm) (Waters)

Flow rate: 0.8 mL/min

UV detection wavelength: 254 nm

Mobile phase: [A]: a 0.1% formic acid-containing aqueous solution, [B]: a 0.1% formic acid-containing acetonitrile solution

Gradient: a linear gradient of 5% to 100% solvent [B] was carried out in 3.5 minutes, and 100% solvent [B] was kept for 0.5 minutes.

### Example 1

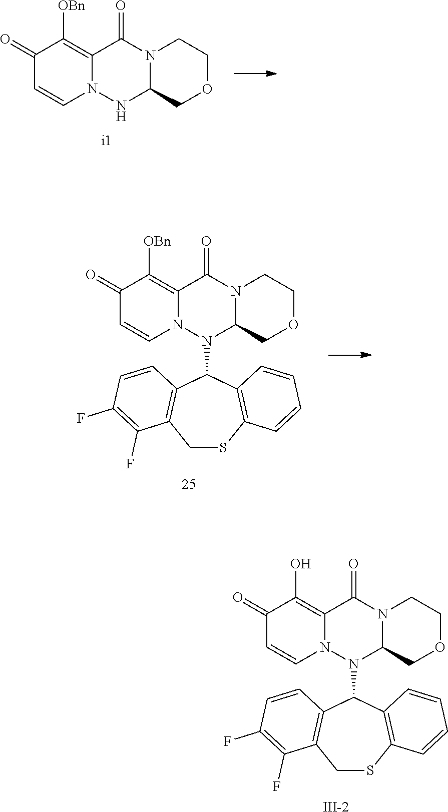

### First Step

Compound i1 (1100 g, 3360 mmol), which is described in WO2016175224 and 7,8-difluoro-6,11-dihydrodibenzothiepine-11-ol (977 g, 3697 mmol) were suspended in 50 wt % T3P in ethyl acetate (3208 g, 5041 mmol) and ethyl acetate (1.1 L). To the mixture was added methanesulfonic acid (436 ml, 6721 mmol) at room temperature and the mixture was stirred at 70° C. for 5.5 hours. To the mixture was added water under ice-water bath and the mixture was stirred at room temperature for 1 hour. THF was added thereto and the mixture was extracted with ethyl acetate. The obtained organic layer was washed with water and 8% aqueous solution of sodium hydrogen carbonate, dried over anhydrous sodium sulfate, and concentrated under reduced pressure. The obtained residue was dissolved in THF (5.5 L) and potassium carbonate (790 g, 5713 mmol) was added thereto. The mixture was warmed up to 50° C., benzyl bromide (240 ml, 2016 mmol) was added dropwise thereto, and the mixture was stirred at 60° C. for 8.5 hours. To the mixture was added dropwise 2 mol/L aqueous solution of hydrochloric acid under ice-water bath, and the mixture was stirred at room temperature for 10 minutes and extracted with ethyl acetate. The obtained organic layer was washed with water and 8% aqueous solution of sodium hydrogen carbonate and dried over anhydrous magnesium sulfate. An activated carbon (Norit SX-2, 240 g) was added thereto, the mixture was filtered through Celite, and the filtrate was concentrated under reduced pressure To the obtained residue was added ethyl acetate and hexane and the presipitated solid was filtered to obtain Compound 25 (1019 g, 1776 mmol, 53%).

¹H-NMR (CDCl₃) δ: 2.88 (1H, t, J=11.2 Hz), 3.28-3.39 (2H, m), 3.72 (1H, d, J=12.6 Hz), 3.86 (1H, d, J=9.6 Hz), 4.03 (1H, d, J=13.9 Hz), 4.45 (1H, d, J=8.6 Hz), 4.67 (1H, d, J=13.1 Hz), 5.19-5.26 (2H, m), 5.45 (1H, d, J=10.9 Hz), 5.63 (1H, d, J=10.9 Hz), 5.77 (1H, d, J=7.6 Hz), 6.40 (1H, d, J=7.8 Hz), 6.68 (1H, t, J=6.9 Hz), 6.94-7.01 (2H, m), 7.03-7.12 (3H, m), 7.29-7.38 (3H, m), 7.61 (2H, d, J=7.1 Hz).

### Second Step

To a solution of Compound 25 (1200 g, 2092 mmol) in DMA (3.6 L) was added lithium chloride (443 g, 10.5 mol) at room temperature, and the mixture was stirred at 80° C. for 3 hours. To the mixture were added acetone (1.2 L), 0.5 mol/L aqueous solution of hydrochloric acid (6.0 L) and water (2.4 L) under ice-water bath, and the mixture was stirred for 1 hour. The presipitated solid was filtered. The obtained solid was dissolved in chloroform, isopropyl ether was added thereto, and the presipitated solid was filtered to obtain Compound 111-2 (950 g, 1965 mmol, 94%).

¹H-NMR (CDCl₃) δ: 2.99 (1H, dt, J=17.5, 6.8 Hz), 3.47 (1H, td, J=11.9, 2.5 Hz), 3.60 (1H, t, J=10.6 Hz), 3.81 (1H, dd, J=11.9, 3.3 Hz), 3.96 (1H, dd, J=11.0, 2.9 Hz), 4.07 (1H, d, J=13.8 Hz), 4.58 (1H, dd, J=10.0, 2.9 Hz), 4.67 (1H, dd, J=13.5, 1.9 Hz), 5.26-5.30 (2H, m), 5.75 (1H, d, J=7.8 Hz), 6.69 (1H, d, J=7.7 Hz), 6.83-6.87 (1H, m), 6.99-7.04 (2H, m), 7.07-7.15 (3H, m).

### Example 2

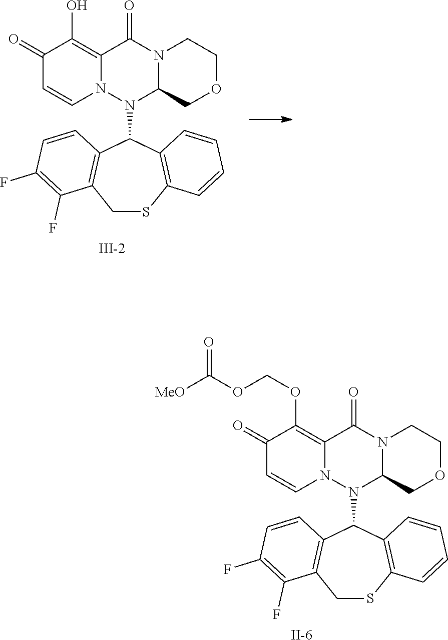

To a suspension of Compound 111-2 (1.00 g, 2.07 mmol) in DMA (5 ml) were added chloromethyl methyl carbonate (0.483 g, 3.10 mmol), potassium carbonate (0.572 g, 4.14 mmol) and potassium iodide (0.343 g, 2.07 mmol) and the mixture was stirred at 50° C. for 6 hours. To the mixture was added DMA (1 ml) and the mixture was stirred for 6 hours. The mixture was cooled to room temperature, DMA (6 ml) was added thereto, and the mixture was stirred at 50° C. for 5 minutes. The mixture was filtered. To the obtained filtrate were added 1 mol/L aqueous solution of hydrochloric acid (10 ml) and water (4 ml) and the mixture was stirred for 1 hour. The presipitated solid was filtered and dried under reduced pressure at 60° C. for 3 hours to obtain Compound II-6 (1.10 g, 1.93 mmol, 93%).

1H-NMR (DMSO-D6) δ: 2.91-2.98 (1H, m), 3.24-3.31 (1H, m), 3.44 (1H, t, J=10.4 Hz), 3.69 (1H, dd, J=11.5, 2.8 Hz), 3.73 (3H, s), 4.00 (1H, dd, J=10.8, 2.9 Hz), 4.06 (1H, d, J=14.3 Hz), 4.40 (1H, d, J=11.8 Hz), 4.45 (1H, dd, J=9.9, 2.9 Hz), 5.42 (1H, dd, J=14.4, 1.8 Hz), 5.67 (1H, d, J=6.5 Hz), 5.72-5.75 (3H, m), 6.83-6.87 (1H, m), 7.01 (1H, d, J=6.9 Hz), 7.09 (1H, dd, J=8.0, 1.1 Hz), 7.14-7.18 (1H, m), 7.23 (1H, d, J=7.8 Hz), 7.37-7.44 (2H, m).

### Example 3

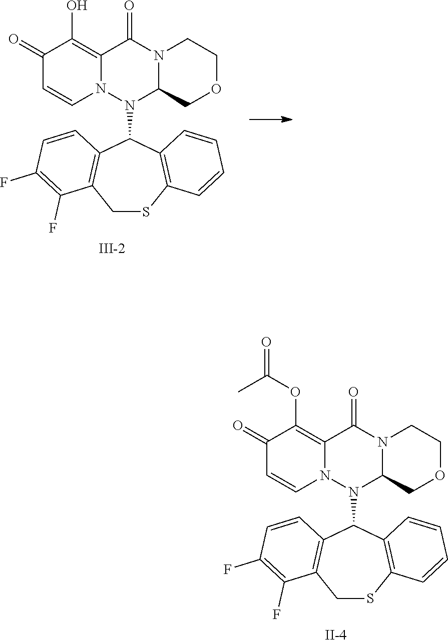

To a solution of Compound III-2 (90 mg, 0.186 mmol) in dichloromethane (2 mL) were added acetic anhydride (0.053 mL, 0.558 mmol), triethylamine (0.077 mL, 0.558 mmol) and a catalytic amount of DMAP, and the mixture was stirred at room temperature for 2 hours. The mixture was concentrated under reduced pressure and the obtained residue was purified by silica gel column chromatography (chloroform-methanol). To the obtained solution was added ether and the presipitated solid was filtered to obtain Compound II-4 (71 mg, 73%).

1H-NMR (CDCl₃) δ:2.46 (s, 3H), 2.88-2.99 (m, 1H), 3.35-3.50 (m, 1H), 3.60-3.65 (m, 1H), 3.75-3.83 (m, 1H), 3.90-4.00 (m, 1H), 4.05 (d, J=14.0 Hz, 1H), 4.52-4.57 (m, 1H), 4.60-4.70 (m, 1H), 5.24-5.34 (m, 1H), 5.35 (s, 1H), 5.88 (d, J=7.6 Hz, 1H), 6.85-6.82 (m, 1H), 6.90-7.05 (m, 2H), 7.06-7.20 (m, 4H)

LC/MS (ESI):m/z=526.2 [M+H]⁺, RT=1.87 min, method (1)

The following example compounds in Table 1 were synthesized from commercially available compounds according to the above examples.

TABLE 1

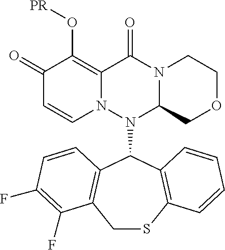

| No. | PR | data | comment |
|---|---|---|---|
| II-5 | 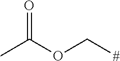 | 1H-NMR(DMSO-d6) δ: 2.04 (s, 3H), 2.90-3.00 (m, 1H) 3.44-3.50 (m, 2H), 3.64-3.72 (m, 1H), 3.95-4.00 (m, 1H) 4.11-4.10 (m, 1H), 4.20- 4.30 (m, 2H), 6.40-5.5.46 (m 1H), 6.62-5.75 (m, 4H), 6.80-6.90 (m,1H), 6.98-7.10 (m, 1H), 7.11-7.20 (m, 2H), 7.21-7.30 (m, 1H), 7.45-7.50 (m, 2H) | |
| II-7 | 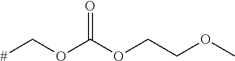 | 1H-NMR(CDCl3) δ: 2.85-2.97 (m, 1H), 3.38 (s. 3H), 3.39-3.48 (m, 1H), 3.54 (t, J = 10.4 Hz, 1H), 3.68 (t, J = 4.4 Hz, 2H), 3.74 (dd, J = 2.8 Hz, 12.0 Hz, 1H). 3.92 (dd, J = 2.8 Hz 10.8 Hz, 1H), 4.05 (d J = 13.6 Hz, 1H), 4.36 (q, J = 4.4 Hz. 2H), 4.51 (dd, J = 2.8 Hz, 9.6 Hz, | |
| | | 1H). 4.65 (d, J = 12.0 Hz, 1H). 5.27 (dd. J = 2.0 Hz, 13.6 Hz, 1H), 5.34 | |
| | | (s, 1H), 5.86 (d, J = 8.0 Hz, 1H), 5.93 (s, 2H), 6.81-6.89 (m, 2H), | |
| | | 6.98-7.15 (m, 5H). | |
| II-8 | 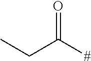 | 1H-NMR (CDCl3) δ: 1.33 (3H. t, J = 7.0 Hz). 2.82 (2H, d, J = 6.1 Hz). 2.93 (1H, t, J = 11.2 Hz), 3.42 (1H, t, J = 11.4 Hz). 3.59 (1H, t, J = 10.2 Hz). 3.78 (1H d, J = 11.2 Hz), 3.96 (1H, d, J = 10.3 Hz), 4.06 (1H, d, J = 13.8 Hz), 4.55 (1H. d, J = 8.9 Hz), 4.63 (1H. d, J = | |
| | | 13.6 Hz), 5.29 (1H, d, J = 13.9 Hz), 5.36 (1H, s). 5.88 (1H, d, J = 7.4 | |
| | | Hz), 6.90 (1H, s), 7.03-7.12 (6H, m). | |
| II-9 | 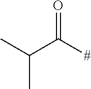 | 1H-NMR (CDCl3) δ: 1.42 (d, J = 6.8 Hz, 6H), 2.85-3.05 (m, 2H), 3.40-3.49 (m, 1H), 3.59 (t, J = 10.4 Hz, 1H), 3.76 (d. J = 11.4 Hz, 1H), 3.94 (d, J = 10.4 Hz, 1H), 4.06 (d. J = 14.1 Hz, 1H), 4.51-4.57 (m, 1H), 4.59-4.70 (m, 1H), 5.25-5.32 (m, 1H), 5.35-5.39 (m 1H), 5.80-5.89 (m, 1H), 6.85-7.15 (m, 7H). | |
| II-10 | 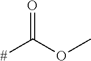 | LC/MS (ESI): m/z = 542 [M + H]+, RT = 1.92 min, method (1) | |
| II-11 | 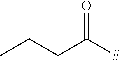 | LC/MS (ESI): m/z = 554 [M + H]+, RT = 2.10 min, method (1) | |

Generally, the disclosed method involves the use of a compound for treating influenza, where the compound is a substituted polycyclic pyridone derivative having cap-dependent endonuclease inhibitory activity, a prodrug thereof, or a pharmaceutical composition including thereof. The disclosed method generally involves administering an effective amount of a compound to a subject having influenza, where the compound is administered initially at least about 48 hours after an onset of influenza.

In one example, the compound includes the substituted polycyclic pyridone derivatives described above.

In one example, the compound has one of the following formulae:

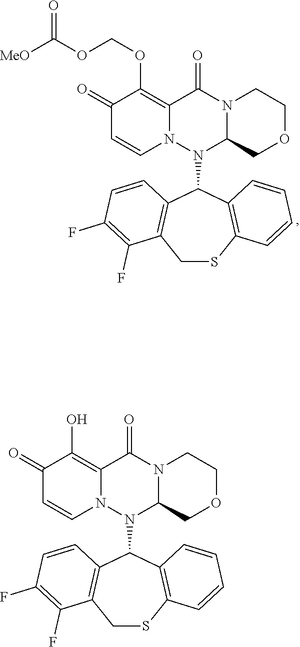 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or its pharmaceutically acceptable salt thereof.

In general, the compound having the formula:

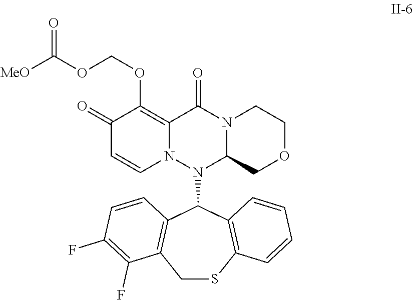 [SMILES: COC(=O)OCOc1c2n(ccc1=O)N([C@@H]1c3ccccc3SCc3c1ccc(F)c3F)[C@@H]1COCCN1C2=O]

can be referred to as baloxavir marboxil or BXM.

In general, the compound having the formula:

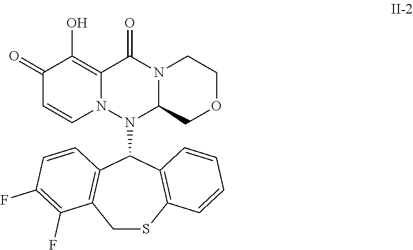 [SMILES: O=C1c2c(O)c(=O)ccn2N([C@@H]2c3ccccc3SCc3c2ccc(F)c3F)[C@@H]2COCCN12]

can be referred to as baloxavir acid or BXA.

In one example, the number of times the compound is administered is not particularly limited. In another example, the compound can be administered only once. In another example, the compound can be administered only two times. In another example, the compound can be administered only three times.

In one example, the onset of influenza in the subject is when the subject has a virus titer sufficient to cause a symptom of influenza to be exhibited in the subject. In one example, the virus titer sufficient to cause a symptom of influenza to be exhibited in the subject is 0.7 log₁₀ TCID₅₀/mL.

In one example, the onset of influenza in the subject is when the subject initially exhibits an increase in a body temperature from a normal temperature of the subject. In another example, the onset of influenza in the subject is when the subject initially exhibits an increase in a body temperature of at least 1° C. from a normal temperature of the subject. In one example, a normal temperature of the subject is based on an average body temperature of the subject before having a virus titer sufficient to cause a symptom of influenza. In one example, the average body temperature is an average of the subject's body temperature measured over a certain time period. In one example, the certain time period is one or more years.

In one example, the onset of influenza in the subject is when the subject initially develops a systemic symptom or a respiratory symptom. In one example, a systemic symptom includes one or more of headache, feverishness, chills, muscular pain, joint pain, and fatigue. In one example, a respiratory symptom includes one or more of coughing, sore throat, and nasal congestion.

In one example, the effective amount of the compound is sufficient to alleviate a symptom of influenza in the subject as compared to a symptom that the subject has when the compound is first administered to the subject.

The phrase “alleviate a symptom of influenza” refers to a self-evaluation of the subject's influenza symptoms using a 4-point scale [0: none, 1: mild, 2: moderate, 3: severe] starting from the time the compound is initially administered. Seven influenza symptoms are evaluated, which are cough, sore throat, headache, nasal congestion, feverishness or chills, muscular or joint pain, and fatigue. Alleviation occurs when all seven influenza symptoms (cough, sore throat, headache, nasal congestion, feverishness or chills, muscular or joint pain, fatigue) become “0: none” or “1: mild”, and this condition continues at least for 21.5 hours (24 hours—10%).

In one example, the effective amount is sufficient to reduce an amount of the virus in the subject as compared to an amount of the virus that the subject has when the compound is first administered to the subject. In one example, the amount of virus in the subject is reduced by about 1/100 to about 1/1000000, alternately about 1/1000 to about 1/1000000, alternately, about 1/10000 to about 1/1000000, alternately about 1/100000 to about 1/1000000, as compared to an amount of the virus that the subject has when the compound is first administered to the subject.

In one example, the effective amount of the compound is in a range from at or about 0.1 mg to at or about 3000 mg. In another example, the effective amount of the compound is in a range from about at or 0.1 to at or about 240 mg. In another example, the effective amount of the compound is in a range from about at or about 5 mg to at or about 80 mg. In yet another example, the effective amount of the compound is in a range from at or about 40 mg to at or about 80 mg. In yet another example, the effective amount is in a range from at or about 10 mg to at or about 80 mg per dose.

In one example, the compound is administered at or before about 120 hours after the onset of the disease in the subject. In one example, the compound is administered at or before about 72 hours, alternately at or before about 84 hours, alternately at or before about 96 hours, alternately at or before about 120 hours, alternately at or before about 144 hours, alternately at or before about 168 hours, after the onset of the disease in the subject. In preferred example, the compound is administered at or before about 96 hours after the onset of the disease in the subject. In another preferred example, the compound is administered at or before about 84 hours after the onset of the disease in the subject.

In one example, the subject is a human patient. In one example, the subject is not a patient who requires hospitalization for severe influenza. In one example, the subject is not a patient who requires an extension of hospitalization because of influenza infection during the hospitalization. In one example, an extension of hospitalization means an extended period of hospitization beyond what was originally prescribed by the hospital.

In one example, the subject is not a patient who has at least one of severity and complication risk factors. In one example, the phrase “severity and complication risk factors” means the presence of at least one of the following factors:

a. Asthma or chronic lung disease (e.g. chronic obstructive pulmonary disease or cystic fibrosis)
b. Endocrine disorders
c. Residents of long-term care facilities (e.g. nursing homes)
d. Compromised immune system
e. Neurological and neurodevelopmental disorders
f. Heart disease (e.g. congenital heart disease, congestive heart failure, or coronary artery disease)
g. Adults aged ≥65 years
h. Blood disorders (e.g. sickle cell disease)
i. Metabolic disorders (e.g. inherited metabolic disorders and mitochondrial disorders)
j. Morbid obesity (e.g. body mass index≥40)
k. Women who are within 2 weeks postpartum and are not breastfeeding

In one example, the subject is not a patient who requires hospitalization for severe influenza or who requires an extension of hospitalization because of influenza infection during the hospitalization. The term “severe influenza” means at least one of the following influenza symptoms:
a. a symptom which requires ventilation or supplemental oxygen to support respiration, and
b. a symptom which is accompanied by complication related to influenza that requires hospitalization (e.g. pneumonia, CNS involvement, myositis, rhabdomyolysis, acute exacerbation of chronic kidney disease, asthma or chronic obstructive pulmonary disease [COPD], severe dehydration, myocarditis, pericarditis, exacerbation of ischemic heart disease).

In one example, the compound is administered based on the weight of the subject. In one example, the compound can be administered as a weight-based dose. In one example, at or about 40 mg is administered to a subject weighing about 40 to under about 80 kg. In one example, about 80 mg is administered to a subject weighing above at or above 80 kg. In one example, the compound is administered on the first day of administration and three days after the first day of administration. In one example, the compound is administered six days after the first day of administration if improvement has not occurred four days after the first day of administration. In some examples, improvement means a lower score in seven of the influenza symptoms (cough, sore throat, headache, nasal congestion, feverishness or chills, muscular or joint pain, and fatigue) using a 4-point scale [0: none, 1: mild, 2: moderate, 3: severe] relative to the time the compound is initially administered.

In one example, the compound is administered orally. In another example, the compound is administered parenterally.

In one example, the compound is administered through at least one route selected from the group consisting of orally, dermally, subcutaneously, intravenously, intraarterially, intramuscularly, intraperitoneally, transmucosally, via inhalation, transnasally, ophthalmically, via an inner ear and vaginally.

Generally, the compound can be administered with any material in any amounts that are suitable for use with the compound. In one example, the compound is administered in combination with at least one material selected from the group consisting of a neuraminidase inhibitor, an RNA-dependent RNA polymerase inhibitor, an M2 protein inhibitor, a PB2 Cap binding inhibitor, a HA maturation inhibitor, a recombinant sialidase, a re-assemble inhibitor, RNA interference compound, a receptor of hemagglutinin binding inhibitor, a membrane of HA fusion inhibitor, a NP nuclear translocation inhibitor, a CXCR inhibitor, a CRM1 inhibitor, an anti-HA antibody and an immunological agent.

In one example, the compound is administered in combination with one or more of oseltamivir, zanamivir, peramivir, laninamivir, favipiravir, amantazine, flumazine,

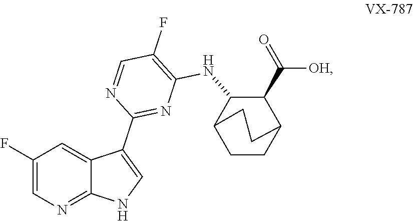

MHAA4549A (as described in McBride et al., Antimicrobial Agents and Chemistry, Vol. 61, Issue 11, (2017)), TCN-032 (as described in Ramos et al., JID 2015:11 (2015)), VIS-410 (as described in Tharakaraman et al., PNAS, vol. 112, no. 35, 10890-10895 (2015)), CR-8020 (as described in Ekiert et al., Science, 333(6044), 843-850 (2011)), CR-6261 (as described in Ekiert et al., Science, 324(5924), 246-251 (2009)), CT-P27 (as described in Celltrion, Press Release, Oct. 12, 2016) and MEDI-8852 (as described in Cell, 166(3), 596-608 (2016)).

In one example, the compound is administered in at least one form selected from the group consisting of a tablet, powder, a granule, a capsule, a pill, a film, a suspension, an emulsion, an elixir, a syrup, lemonade, spirit, aromatic water, extract, decoction and tincture.

In one example, the compound is administered in at least one form selected from the group consisting of a sugar-coated tablet, a film-coated tablet, an enteric-coated tablet, a sustained-release tablet, a troche tablet, a sublingual tablet, a buccal tablet, a chewable tablet, an orally disintegrated tablet, a dry syrup, a soft capsule, a micro capsule or a sustained-release capsule.

In one example, the compound is administered in at least one form selected from the group consisting of an injection, an infusion, an eye drop, a nose drop, an ear drop, an aerosol, an inhalation, a lotion, an impregnation, a liniment, a mouthwash, an enema, an ointment, a plaster, a jelly, a cream, a patch, a cataplasm, an external powder or a suppository.

### Test Example 1: Measurement of Cap-Dependent Endonuclease (CEN) Inhibitory Activity

### 1) Preparation of Substrate

30merRNA(5′-pp[m2′-O]GAA UAU(-Cy3) GCA UCA CUA GUA AGC UUU GCU CUA-BHQ2-3′: manufactured by Japan Bio Services Co., LTD.) in which G at a 5′ end is diphosphate-modified, a hydroxy group at 2′ position is methoxylation-modified, U sixth from a 5′ end is labelled with Cy3, and a 3′ end is labelled with BHQ2 was purchased, and a cap structure was added using ScriptCap system manufactured by EPICENTRE (a product was m7G [5′]-ppp-[5′] [m2′-O]GAA UAU(-Cy3) GCA UCA CUA GUA AGC UUU GCU CUA(-BHQ2)-3′). This was separated and purified by denatured polyacrylamide gel electrophoresis, and used as a substrate.

### 2) Preparation of Enzyme

RNP was prepared from a virus particle using standard method (Reference Document: VIROLOGY (1976) 73, p 327-338 OLGA M. ROCHOVANSKY). Specifically, A/WSN/33 virus (1×10³ PFU/mL, 200 μL) was inoculated in a 10 days old embryonated chicken egg. After incubation at 37° C. for 2 days, the allantoic fluid of the chicken egg was recovered. A virus particle was purified by ultracentrifugation using 20% sucrose, solubilized using TritonX-100 and lysolecithin, and an RNP fraction (50-70% glycerol fraction) was collected by ultracentrifugation using a 30-70% glycerol density gradient, and was used as an enzyme solution (containing approximately 1 nM PB1-PB2-PA complex).

### 3) Enzymatic Reaction

An enzymatic reaction solution (2.5 μL) (composition: 53 mM Tris-hydrochloride (pH 7.8), 1 mM MgCl2, 1.25 mM dithiothreitol, 80 mM NaCl, 12.5% glycerol, enzyme solution 0.15 μL) was dispensed into a 384-well plate made of polypropylene. Then, 0.5 μL of a test compound solution which had been serially diluted with dimethyl sulfoxide (DMSO) was added to the plate. As a positive control (PC) or a negative control (NC), 0.5 μL of DMSO was added to the plate respectively. Each plate was mixed well. Then, 2 μL of a substrate solution (1.4 nM substrate RNA, 0.05% Tween20) was added to initiate a reaction. After room temperature incubation for 60 minutes, 1 μL of the reaction solution was collected and added to 10 μL of a Hi-Di formamide solution (containing GeneScan 120 Liz Size Standard as a sizing marker: manufactured by Applied Biosystems (ABI)) in order to stop the reaction. For NC, the reaction was stopped in advance by adding EDTA (4.5 mM) before initiation of the reaction (all concentrations described above are final concentrations).

### 4) Measurement of Inhibition Ratio (IC₅₀ Value)

The solution for which the reaction was stopped was heated at 85° C. for 5 minutes, rapidly cooled on ice for 2 minutes, and analyzed with an ABI PRIZM 3730 genetic analyzer. A peak of the cap-dependent endonuclease product was quantitated by analysis software ABI Genemapper, a CEN reaction inhibition ratio (%) of a test compound was obtained by setting fluorescent intensities of PC and NC to be 0% inhibition and 100% inhibition, respectively, an IC₅₀ value was obtained using curve fitting software (XLfit2.0: Model 205 (manufactured by IDBS) etc.).

### (Result)

Compund 111-2: CEN IC₅₀=1.93 nM

### Test Example 2: CPE Inhibitory Effect Confirming Assay

### <Material>

2% FCS E-MEM (prepared by adding kanamycin and FCS to MEM (Minimum Essential Medium) (Invitrogen))
0.5% BSA E-MEM (prepared by adding kanamycin and BSA to MEM (Minimum Essential Medium) (Invitrogen))
HBSS (Hanks' Balanced Salt Solution)
MDBK cell
   Cells were adjusted to the appropriate cell number (3×10⁵/mL) with 2% FCS E-MEM.
MDCK cell
   After washing with HBSS two times, cells were adjusted to the appropriate cell number (5×10⁵/mL) with 0.5% BSA E-MEM.
Trypsin solution
   Trypsin from porcine pancreas (SIGMA) was dissolved in PBS(−), and filtrated with a 0.45 μm filter.
EnVision (PerkinElmer)
WST-8 Kit (Kishida Chemical Co., Ltd.)
10% SDS solution

### <Operation Procedure>

Dilution and Dispensation of Test Sample

As a culture medium, 2% FCS E-MEM was used at the use of MDBK cells, and 0.5% BSA E-MEM was used at the use of MDCK cells. Hereinafter, for diluting virus, cells and a test sample, the same culture medium was used.

A test sample was diluted with a culture medium to an appropriate concentration in advance, and then 2 to 5-fold serial dilution on a 96 well plate (50 μL/well) was prepared. Two plates, one for measuring anti-Flu activity and the other for measuring cytotoxity, were prepared. Each assay was performed triplicate for each drug.

At the use of MDCK cells, Trypsin was added to the cells to be a final concentration of 3 μg/mL only for measuring anti-Flu activity.

Dilution and Dispensation of Influenza Virus

An influenza virus was diluted with a culture medium to an appropriate concentration in advance, and each 50 μL/well was dispensed on a 96-well plate containing a test substance. Each 50 μL/well of a culture medium was dispensed on a plate containing a test substance for measuring cytotoxity.

Dilution and Dispensation of Cell

Each 100 μL/well of cells which had been adjusted to the appropriate cell number was dispensed on a 96 well plate containing a test sample.

This was mixed with a plate mixer, and incubated in a CO2 incubator for 3 days for measuring anti-Flu activity and measuring cytotoxity.

Dispensation of WST-8

The cells in the 96-well plate which had been incubated for 3 days was observed visually under a microscope, and appearance of the cells, the presence or absence of a crystal of test substance were checked. The supernatant was removed so that the cells were not absorbed from the plate.

WST-8 Kit was diluted 10-fold with a culture medium, and each 100 μL was dispensed into each well. After mixing with a plate mixer, cells were incubated in a CO2 incubator for 1 to 3 hours.

After incubation, regarding the plate for measuring anti-Flu activity, each 10 μL/well of a 10% SDS solution was dispensed in order to inactivate a virus.

Measurement of Absorbance

After the 96-well plate was mixed, absorbance was measured with EnVision at two wavelengths of 450 nm/620 nm.

### <Calculation of Each Measurement Item Value>

The value was calculated using Microsoft Excel or a program having the equivalent calculation and processing ability, based on the following calculation equation.

Calculation of Effective Inhibition Concentration to Achieve 50% Influenza Infected Cell Death (EC₅₀)

EC₅₀=10^{Z}

Z=(50%−High %)/(High %−Low %)×{log(High conc.)−log(Low conc.)}+log(High conc.)

### (Result)

Compund III-2: CPE EC₅₀=1.13 nM

Based on the above results, the parent compound exhibits high cap-dependent endonuclease (CEN) inhibitory activity and/or high CPE inhibitory effect and thus can be a useful agent for treatment and/or prevention of symptom and/or disease induced by infection with influenza virus.

### Test Example 3: BA Test

### Materials and Methods for Experiments to Evaluate Oral Absorption

(1) Experimental animals: mice or SD rats were used.
(2) Rearing condition: mice or SD rats were fasted and were allowed free access to sterilized tap water.
(3) Setting of dosage and grouping: Oral administration and intravenous administration were performed with the predetermined dosage. Grouping was set as below. (Dosage was changed per compound)

Oral administration 1 to 30 mg/kg (n=2 to 3)

Intravenous administration 0.5 to 10 mg/kg (n=2 to 3)

(4) Preparation of administration solutions: Oral administration was performed as solution or suspension. Intravenous administration was performed after solubilization.
(5) Routes of administration: Oral administration was performed mandatory into the stomach by oral sonde. Intravenous administration was performed from caudal vein by syringes with needle.
(6) Evaluation items: Blood was collected serially and concentration of a compound used in the present invention in plasma was measured by LC/MS/MS.
(7) Statistical analysis: About transition of concentration of a compound used in the present invention in plasma, the area under the plasma concentration versus time curve (AUC) was calculated by non-linear least-squares method program, WinNonlin (a registered trademark), and bioavailability (BA) of a compound used in the present invention was calculated from AUCs of the oral administration group and the intravenous administration group. The BAs of each compound are described in Table 2 below.

### (Result)

TABLE 2

| No. | BA(%) |
|---|---|
| II-4 | 20.0 |
| II-5 | 17.8 |
| II-6 | 14.9 |
| II-7 | 14.5 |
| II-8 | 27.8 |
| II-9 | 15.0 |
| II-10 | 10.6 |
| II-11 | 11.0 |
| III-2 | 4.2 |

Based on the above results, the prodrug had improved bioavailability rather than the parent compound.

Therefore, the compound used in the present invention has excellent oral absorbability and can be a useful agent for treatment and/or prevention of symptom and/or disease induced by infection with influenza virus.

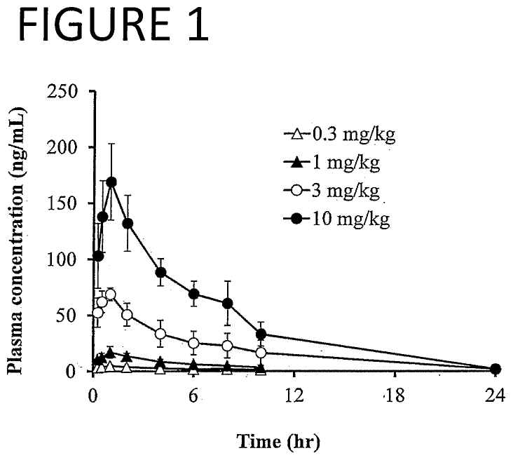

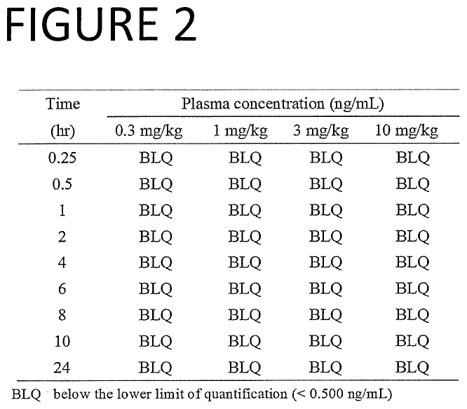

FIGS. 1 and 2 show a result of measuring the plasma concentration of Compound III-2 and Compound II-6 after oral administration of prodrug Compound II-6, the parent compound of which is Compound III-2, to rat under non-fasting conditions.

In addition, the concentration of Compound II-6 in all plasma samples was a determination limit or less. Therefore, prodrug Compound II-6, the parent compound of which is Compound III-2 is found to have changed promptly to Compound III-2 in vivo after administration (see FIG. 2).

Based on the above test results, it was revealed that the compound converted into a prodrug was absorbed into the body after oral administration, and rapidly converted into a parent compound in the blood. Therefore, the compound used in the present invention can be a useful agent for treatment and/or prevention of symptom and/or disease induced by infection with influenza virus.

### Formulation Example

The following Formulation Examples are only exemplified and not intended to limit the scope of the invention.

### Formulation Example 1: Tablets

The compounds used in the present invention, lactose and calcium stearate are mixed. The mixture is crushed, granulated and dried to give a suitable size of granules. Next, calcium stearate is added to the granules, and the mixture is compressed and molded to give tablets.

### Formulation Example 2: Capsules

The compounds used in the present invention, lactose and calcium stearate are mixed uniformly to obtain powder medicines in the form of powders or fine granules. The powder medicines are filled into capsule containers to give capsules.

### Formulation Example 3: Granules

The compounds used in the present invention, lactose and calcium stearate are mixed uniformly and the mixture is compressed and molded. Then, it is crushed, granulated and sieved to give suitable sizes of granules.

### Formulation Example 4: Orally Disintegrated Tablets

The compounds used in the present invention and crystalline cellulose are mixed, granulated and tablets are made to give orally disintegrated tablets.

### Formulation Example 5: Dry Syrups

The compounds used in the present invention and lactose are mixed, crushed, granulated and sieved to give suitable sizes of dry syrups.

### Formulation Example 6: Injections

The compounds used in the present invention and phosphate buffer are mixed to give injection.

### Formulation Example 7: Infusions

The compounds used in the present invention and phosphate buffer are mixed to give injection.

### Formulation Example 8: Inhalations

The compound used in the present invention and lactose are mixed and crushed finely to give inhalations.

### Formulation Example 9: Ointments

The compounds used in the present invention and petrolatum are mixed to give ointments.

### Formulation Example 10: Patches

The compounds used in the present invention and base such as adhesive plaster or the like are mixed to give patches.

### Test Example 4: Mouse Model for Delayed Treatment with BXM

(1) Materials and methods

### (1.1) Compounds

BXM was synthesized as described above. Oseltamivir phosphate (OSP) was purchased from Sequoia Research Products (Oxford, UK). The suspension of BXM and solution of OSP were prepared with 0.5% methylcellulose solution (MC).

(1,2) Cells and viruses

Madin-Darby canine kidney (MDCK) cells were obtained from the European Collection of Cell Cultures. A/Puerto Rico (PR)/8/34 strains of influenza virus were obtained from the American Type Culture Collection. For virus quantitation, serial dilutions of lung homogenates were inoculated onto confluent MDCK cells as described previously (Kitano et al., 2013). The presence of cytopathic effects (CPE) was determined under a microscope and virus titers were calculated as log₁₀ 50% tissue culture infectious dose (TCID₅₀)/ml. When no CPE was observed in the lowest dilution, it was defined as an undetectable level that was considered to be lower than 1.5 log₁₀ TCID₅₀/ml.

### (1.3) Animals

Specific-pathogen-free 6-week-old female BALB/c mice (Charles River Laboratories Japan, Inc.) were used in the study. Body weights and survival were monitored daily, and the mice were euthanized when they lost more than 30% of their body weight compared to their weight pre-infection according to humane endpoints. All mouse studies were conducted under applicable laws and guidelines and with the approval of the Shionogi Animal Care and Use Committee.

### (2) Antiviral Study in a Mouse Model

### (2.1) The Effect of Delayed Administration of BXM in Lethal Infection Mouse Model

Mice infected with A/PR/8/34 (1.38×10³ TCID₅₀) were treated orally with BXM at doses of 1.5 and 15 mg/kg twice daily for 5 days from 24, 48, 72, or 96 hours post infection. OSP at a dose of 5 mg/kg was administered orally twice daily for 5 days. Control mice were treated with 0.5% MC for 5 days. Mice were examined daily for survival and body weight through 28 days post infection. To examine the effects of BXM on viral replication in mice when treatment of BXM was initiated at 72 hours post infection, eight mice in each group were euthanized, and then the lungs were removed at days 1, 3, 4, 6, 8, and 10 post infection.

### (3) Statistical Analysis

Differences in survival time after virus infection were analyzed by log rank test. The comparisons of the proportion of body weight at each time point to initial body weight were analyzed by Student's t-test. When the mouse died before evaluated time point, the proportion of body weight of the mouse was regarded as 70%. The one-way analysis of variance model and contrast method by the analysis time points were applied for evaluating the virus titers in the lungs. The fixed-sequence procedure was used to adjust the multiplicity. Statistical analysis was performed using the statistical analysis software, SAS version 9.2 for Windows (SAS Institute, Cary, N.C.). Two-sided adjusted P values below 0.05 were considered as statistically significant.

### (4) Results

### (4.1) Effects of Delayed Administration of BXM Against Lethal Influenza a Virus Infection in Mice.

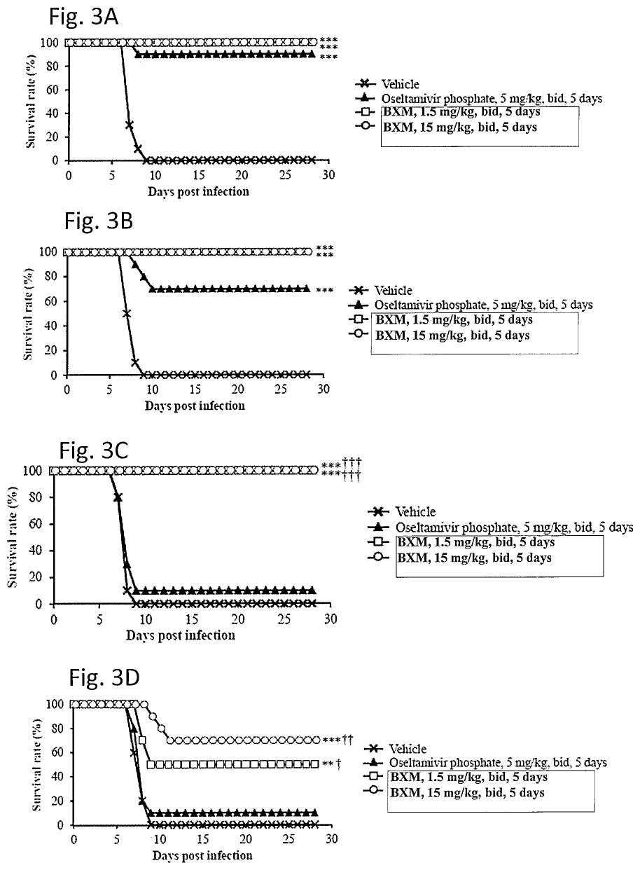

The effects of late administration of BXM in mice lethally infected with influenza A virus were examined. To this end, administration was started with 1.5 and 15 mg/kg of BXM from 24, 48, 72, or 96 hours post infection and the treatment continued for up to 5 days with observation until day 28. All vehicle-treated mice inoculated with A/PR/8/34 (1.38<10³ TCID₅₀) died by day 9 post infection. When treatment of BXM was delayed until 24, 48, or 72 hours post infection, all mice treated with 1.5 and 15 mg/kg of BXM survived (FIGS. 3 A, B, and C). When treatment of BXM was initiated at 96 hours post infection, survival rates of mice treated with 1.5 and 15 mg/kg of BXM were 50% and 70%, respectively (FIG. 3D). In contrast, when OSP treatment was delayed until 24 or 48 hours post infection, the mice survived for a significant longer period than the vehicle-treated group (FIGS. 3 A and B), whereas the protection level was decreased when the treatment started at 72 or 96 hours post infection (FIGS. 3 C and D). Comparing the efficacy of BXM and OSP on survival time for the same starting points of treatment, the survival time in the group given BXM at 72 and 96 hours post infection was significantly prolonged compared with that in OSP-treated group.

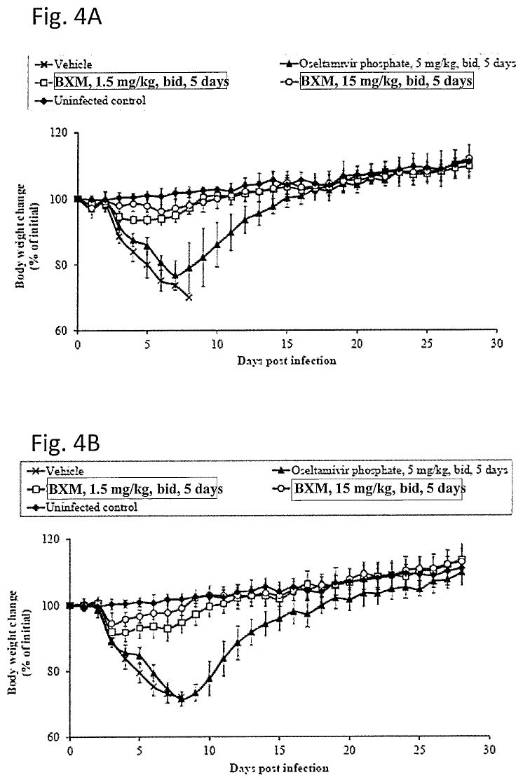

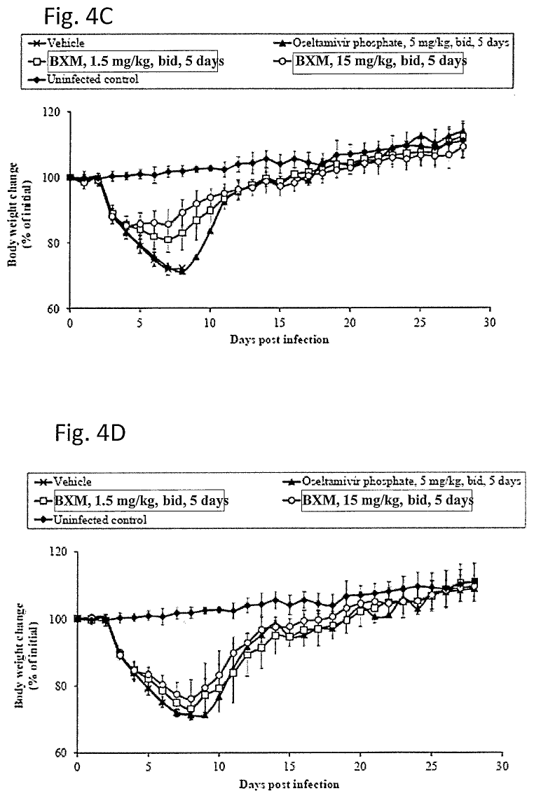

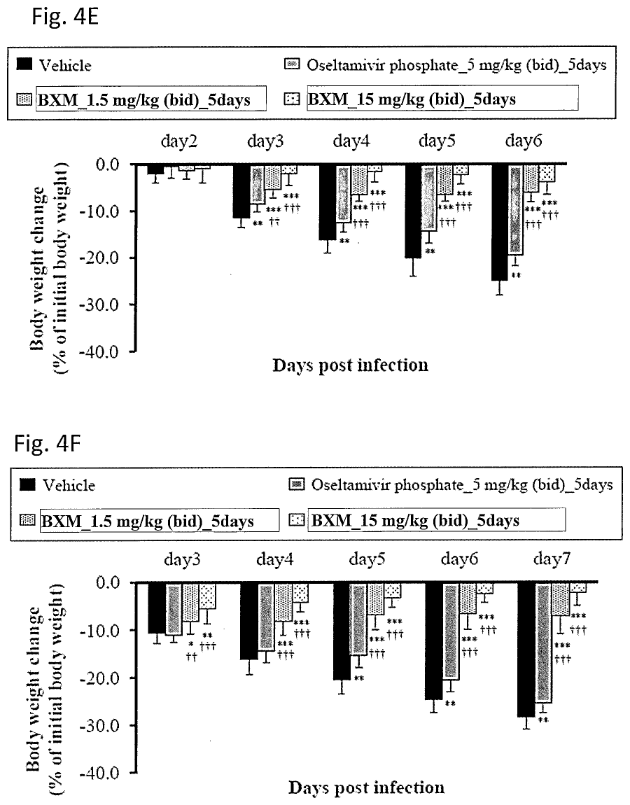

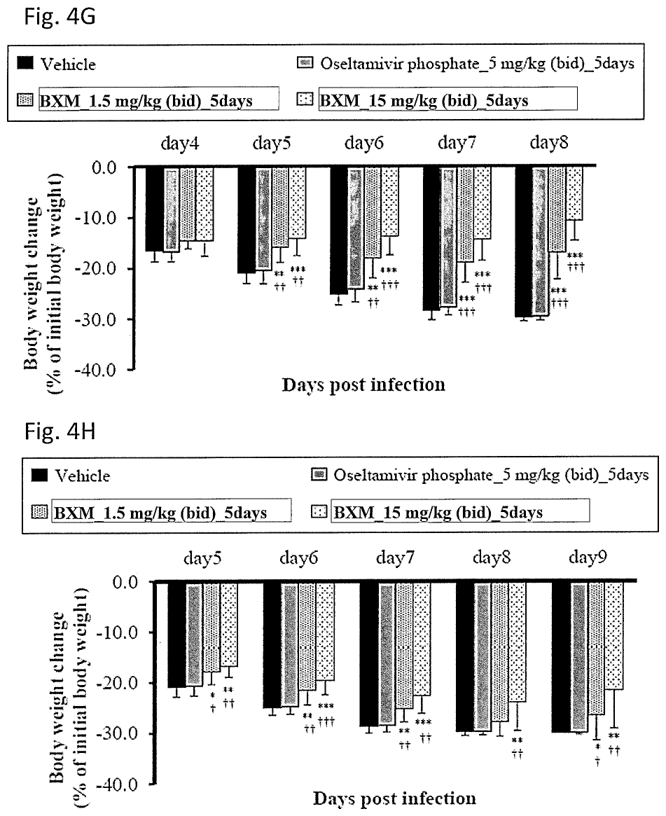

To further characterize the effects of delayed treatment with BXM, we compared body weight change during the treatment period for the same starting points of treatment. All groups treated with BXM from 24, 48, 72, or 96 hours post infection showed significantly less reduction of body weight compared with the vehicle-treated group (FIG. 4). Significant inhibitions in body weight loss were also observed in the groups treated with OSP at 24 and 48 hours post infection compared with the control group, while the groups treated with OSP at 72 and 96 hours post infection showed body weight loss comparable to the vehicle-treated group. These results suggest that BXM may expand the therapeutic window and provide superior therapeutic benefit compared with OSP in mice model.

### (4.2) Inhibitory Effects of Delayed Administration of BXM on Virus Replication in Mice.

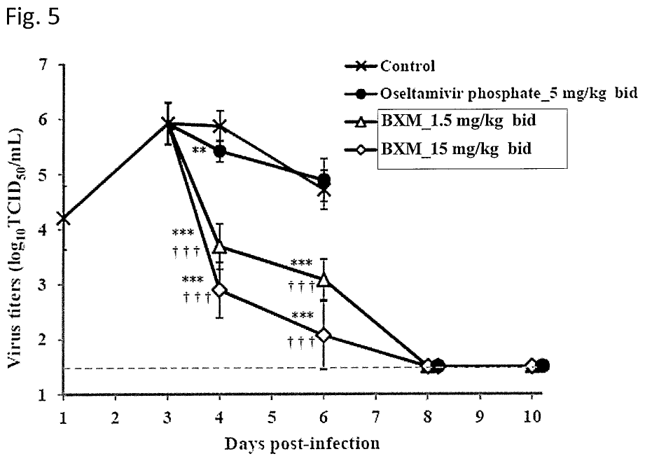

To gain a better understanding of the mechanism by which delayed administration of BXM protect mice from lethal virus infection, we examined the inhibitory effects on virus replication in mouse lung. To do this, the mice inoculated with A/PR/8/34 (1.38×10³ TCID₅₀) were administered 1.5 and 15 mg/kg of BXM for 5 days starting at 72 hours post infection. On days 4 and 6 post infection, virus titers in all groups treated with BXM were significantly reduced compared with those in the vehicle-treated group (FIG. 5). Significant reduction in virus titers was observed in the group treated with 5 mg/kg of OSP compared with vehicle-treated group on day 4 post infection, but not on day 6 post infection. Comparing the efficacy of BXM and OSP, the virus titer on days 4 and 6 post infection in the groups treated with 1.5 and 15 mg/kg of BXM was significantly lower than that in the group treated with 5 mg/kg of OSP.

Previously conducted clinical studies have indicated that administration of NA inhibitors is required to be initiated within 48 hours of the onset of symptoms (see Treanor et al., JAMA February 23:288(8):1016-24 (2000), Dobson et al., Lancet, 385:1729-37 (2015), Sugaya, J. Inect. Chemother. 17, 5950601(2011)). Since no effective treatment is available to patients infected with influenza virus who have not received treatment within 48 hours after its onset, the therapeutic effects of BXM were investigated in a mouse model for delayed treatment. In this study, mice were treated with 5 mg/kg twice daily for 5 days (equivalent to the clinical dose) of OSP as the reference drug treatment (Ward et al., J. Antimicrob Chemother., February:55 Suppl 1:i5-i21 (2005)).

When the treatment with BXM was delayed up to 96 hours post infection, repeated oral administration of BXM was more effective for the prevention of mortality and inhibition of body weight loss than administration of vehicle or OSP in mice infected with influenza A virus. In contrast, when treatment with OSP began at 24 or 48 hours post infection, survival rate of mice was enhanced, but this finding did not occur at 72 or 96 hour post infection. In addition, BXM conferred greater survival benefits than OSP at all treatment initiation times, suggesting that BXM could extend the therapeutic window against influenza virus infection in this mice model. Moreover, when repeated treatment with BXM was initiated at 72 hours post infection, virus titers were significantly lower than those of mice treated with vehicle and OSP. On the other hand, virus titers in lungs of OSP-treated group were comparable to those of the vehicle-treated group. These findings suggested that BXM potently suppressed viral replication and prevented lethality with the treatment initiated not only in the early phase but also in the late phase of infection.

The PK/PD analysis using the plasma concentrations of BXA in the mouse models for infection revealed that the drug concentrations at the end of the dosage interval after the first dosing (C_{τ}) or C₂₄ is a PK parameter that best correlates with the virus titer in the lung 24 hours after the first dose. The C2 of BXA after oral administration of BXM at 15 mg/kg twice a day was 6.85 ng/mL. The C₂₄ (57.1 ng/mL) after administration of BXM in humans treated with the therapeutic dose of 40 mg substantially exceeded the C_{τ} (6.85 ng/mL) in the mouse model (http://www.pmda.go.jp/drugs/2018/P20180312001/index.html). In addition, the simulated C₁₂₀ in plasma concentration of BXA after the single oral dose of BXM in humans at 40 mg is equivalent or higher than the plasma concentration of BXA in mice treated with 15 mg/kg twice daily for 5 days of BXM. Taken together, a dose of BXM at 15 mg/kg twice daily for 5 days is within the therapeutic concentration range achieved by a single dose of 40 mg in humans.

In these studies, BXM at 15 mg/kg twice daily for 5 days eliminated mortality and significantly reduced virus titre 24 hours after administration, whereas clinically equivalent doses of OSP treatments were not as effective. Therefore, it was suggested that there is effectiveness even in patients after 48 hours of onset of influenza, because effectiveness was confirmed in mice treated with oral administration of 15 mg/kg of BXM twice daily for 5 days, which plasma concentration of BXA is lower than that in humans treated with 40 mg of BXM.

### Test Example 5: Clinical Trial

The efficacy and safety of a single oral administration of an investigational drug (BXM: 40 mg, 80 mg) to patients at or after 48 hours of the onset of symptoms by influenza virus are evaluated by a randomized, double-blind comparative study in comparison to 75 mg Oseltamivir administered twice per day for 5 days or a placebo. As for the primary endpoint, subjects make evaluations by themselves on a 4-point scale [0: none, 1: mild, 2: moderate, 3: severe] concerning the time to alleviation of influenza symptoms (the time from the beginning of administration of the investigational drug until 7 influenza symptoms (“cough”, “sore throat”, “headache”, “nasal congestion”, “feverishness or chills”, “muscular or joint pain”, and “fatigue”) were alleviated) to evaluate the efficacy of the investigational drug over the placebo.

Moreover, as for the secondary efficacy endpoint, the efficacy and the side effects of the investigational drug are evaluated according to the influenza virus titer using a nasal or throat swab.

(1) Patients who satisfied all of the following criteria are selected as subjects.
(1.1) Male or female patients at 12 years old or older and younger than 65 years old
(1.2) Patients with a diagnosis of influenza confirmed by all of the following:
a. Fever ≥38° C. (axillary) in the predose examinations or >4 hours after dosing of antipyretics if they were taken
b. At least one of the following general systemic symptoms associated with influenza are present with a severity of moderate or greater
Headache
Feverishness or chills
Muscle or joint pain
Fatigue
   c. At least one of the following respiratory symptoms associated with influenza are present with a severity of moderate or greater
Cough
Sore throat
Nasal congestion
   (1.3) Patients at or after 48 hours of the onset of symptoms
   The definition of onset is any of the following.
   a. Time of the first increase in body temperature (an increase of at least 1° C. from normal body temperature)
   b. Time when the patient experiences at least one general or respiratory symptom
   (2) Method for administering investigational drug
   (i) Test drug

### 20 mg Tablet of BXM

(ii) Placebo or control drug
Placebo for 20 mg tablet of BXM

### 75 mg Capsule of Oseltamivir

Placebo for 75 mg capsule of Oseltamivir
(3) Dosage and administration method

Eligible patients at 12 to 64 years old are randomly allocated to a group receiving a single administration of BXM (40 or 80 mg depending on the body weight), a group receiving 75 mg Oseltamivir twice a day for 5 days, and a placebo group in a ratio of 2:2:1.

The dosage of BXM is 40 mg for subjects weighing less than 80 kg, and 80 mg for subjects weighing 80 kg or more.

(4) Investigational drug for each administered group
[BXM group]

### Day 1:

20 mg Tablets of BXM are administered orally (2 tablets or 4 tablets depending on the body weight). Placebo capsules for Oseltamivir are administered orally twice a day (morning, evening), one capsule per administration.

### Day 2 to Day 5:

Placebo capsules for Oseltamivir are administered orally twice a day (morning, evening), one capsule per administration.

### [Oseltamivir Group]

### Day 1:

Placebo tablets for BXM are administered orally (2 tablets or 4 tablets depending on the body weight). 75 mg Capsules of Oseltamivir are administered orally twice a day (morning, evening), one capsule per administration.

### Day 2 to Day 5:

75 mg Capsules of Oseltamivir are administered orally twice a day (morning, evening), one capsule per administration.

### [Placebo Group]

### Day 1:

Placebo tablets for BXM are administered orally (2 tablets or 4 tablets depending on the body weight). Placebo capsules for Oseltamivir were administered orally twice a day (morning, evening), one capsule per administration.

### Day 2 to Day 5:

Placebo capsules for Oseltamivir were administered orally twice a day (morning, evening), one capsule per administration.
“Day 1” indicates the first day of administration, and “Day 2 to Day 5” indicates the second day to the fifth day as counted from the first day of administration.

### (5) Main Efficacy Endpoint

The main efficacy endpoint is the time to alleviation of influenza symptoms (the time to alleviation of influenza symptoms).

It is the time from the beginning of administration until alleviation of influenza symptoms. Alleviation of influenza symptoms refers to when all 7 influenza symptoms (cough, sore throat, headache, nasal congestion, feverishness or chills, muscular or joint pain, fatigue) become “0: none” or “1: mild” in the patient diary that the subject keeps, and this condition continues at least 21.5 hours (24 hours—10%).

(6) Secondary efficacy endpoint

The secondary efficacy endpoint is as follows.

(6.1) Proportion of patients having a positive influenza virus titer at various points
(6.2) Amount of change in virus titer from baseline at various points
(6.3) Time to termination of viral shedding based on virus titer
(6.4) Incidence of side effects
(7) The virus titer is measured in the following manner.
(7.1) MDCK-SIAT1 cells seeded in a flat-bottom 96-well microplate are cultured in a 5% CO2 incubator at 37±1° C. for 1 day.
(7.2) A standard strain (influenza virus AH3N2, A/Victoria/361/2011, storage condition: −80° C., origin: National Institute of Infectious Diseases), a sample (collected from patients in Phase III clinical test of BXM and stored in an ultra-low-temperature freezer), and a medium for cell control are diluted 101 to 107 folds by a 10-fold serial dilution method.
(7.3) After cells present in a sheet form are confirmed under an inverted microscope, the medium was removed, and a new medium is added at 100 μL/well.
(7.4) The medium is removed.
(7.5) Each of the samples (100 to 107) prepared in (2) above is inoculated at 100 μL/well, using 4 wells per sample.
(7.6) Centrifugal adsorption is performed at room temperature at 1000 rpm for 30 minutes.
(7.7) After centrifugation, the medium is removed, and cells were washed once with a new medium.
(7.8) A new medium is added at 100 μL/well.
(7.9) Incubation is performed in a 5% CO2 incubator at 33±1° C. for 3 days.
(7.10) After incubation, the CytoPathic Effect (CPE) is evaluated under an inverted microscope.
(8) Method for determining to have a positive virus titer

When the detection limit is exceeded as measured by the above virus titer measurement method, it is determined to be positive.

(9) Analysis of primary endpoint

As for the time to alleviation of influenza symptoms, which is the primary endpoint, the primary analysis and the secondary analysis are described. The primary analysis is performed on the ITTI group.

(9.1) Primary analysis

For patients at 12 to 64 years old, the placebo group and the investigational drug administered group are compared by stratified generalized Wilcoxon test using the total score of 7 influenza symptoms before administration (11 points or less, 12 points or more) and regions (Japan/Asia, other regions) as stratification factors.

Also, a Kaplan-Meier survival curve is drawn for each group to calculate the median time to alleviation of influenza symptoms and the 95% confidence interval thereof as well as the difference between the groups in the time to alleviation of influenza symptoms and the 95% confidence interval thereof.

(10) Analysis of secondary endpoint

The following secondary efficacy endpoints are compared between the BXM group and the placebo group and between the BXM group and the Oseltamivir group.

(10.1) Proportion of patients having a positive influenza virus titer at various time points

Only the patients having a virus titer equal to or greater than the determination limit before the beginning of administration in Visit 1 are included in the analysis. In each Visit, a Mantel-Haenszel test using the total score of 7 influenza symptoms before administration and the regions as stratification factors is applied, and the proportion of patients having a positive virus titer is compared between two groups.

(10.2) Amount of change in virus titer from baseline at various time points

Only the patients having a virus titer before the beginning of administration in Visit 1 are included in the analysis. In each Visit, a van Elteren test using the total score of 7 influenza symptoms before administration and the regions as stratification factors is applied, and the amount of change in influenza virus titer from the baseline is compared between two groups.

(10.3) Time to termination of viral shedding based on virus titer

Only the patients having a virus titer equal to or greater than the determination limit before the beginning of administration in Visit 1 are included in the analysis. A stratified generalized Wilcoxon test using the total score of 7 influenza symptoms before administration and the regions as stratification factors is applied.

(10.4) Incidence of side effects

The number of side-effect episodes and the number of patients with side effect are counted for each administration group.

### Aspects

1. A method for treating influenza, comprising:
administering an effective amount of a compound to a subject having an influenza virus,
wherein the compound is administered initially about 48 hours after an onset of influenza in the subject, and
wherein the compound is represented by the following formulae:

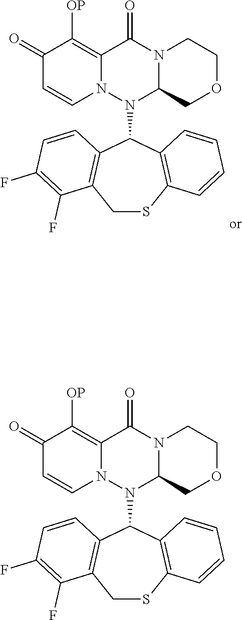

wherein P is hydrogen or a group to form a prodrug, or its pharmaceutically acceptable salt.
2. The method of any one of the above aspects,
wherein the group to form a prodrug is a group selected from the following formula:

—C(═O)—P^{R0}, a)

—C(═O)—O—P^{R2}, g)

—C(═O)—O-L-O—P^{R2}, i)

—C(P^{R3})₂—O—C(═O)—P^{R4}, l)

—C(P^{R3})₂—O—C(═O)—O—P^{R4}, and m)

—C(P^{R3})₂-O—C(═O)—O-L-O—P^{R4} o)

wherein L is straight or branched lower alkylene;
P^{R0} is alkyl;
P^{R2} is alkyl;
P^{R3} is each independently hydrogen; and
P^{R4} is alkyl.
3. A method for treating influenza, comprising:
administering an effective amount of a compound to a subject having an influenza virus,
wherein the compound is administered about 48 hours after an onset of influenza in the subject, and
wherein the compound is represented one of the following formula:

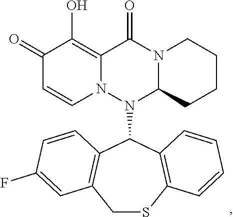 [SMILES: O=C1c2c(O)c(=O)ccn2N([C@H]2c3ccc(F)cc3CSc3ccccc32)[C@@H]2CCCCN12]

or its pharmaceutically acceptable salt.
4. A method for treating influenza, comprising:
administering an effective amount of a compound to a subject having an influenza virus,
wherein the compound is administered initially at least about 48 hours after an onset of influenza in the subject, and
wherein the compound has one of the following formulae:

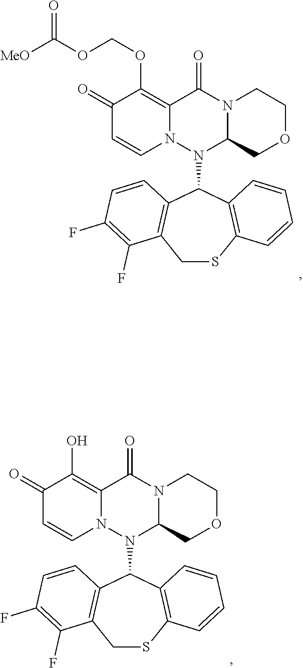 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or its pharmaceutically acceptable salt thereof.
5. The method of any one of the above aspects, wherein the effective amount of the compound is in a range from about 0.1 to about 240 mg.
6. The method of any one of the above aspects, wherein the effective amount of the compound is in a range from about 5 to about 80 mg.
7. The method of any one of the above aspects, wherein the effective amount of the compound is in a range from about 40 to about 80 mg.
8. The method of any one of the above aspects, wherein the effective amount of the compound is in a range from about 10 to about 80 mg per dose.
9. The method of any one of the above aspects, wherein the compound is administered only one time.
10. The method of any one of the above aspects, wherein the compound is administered only one time, two times or three times.
11. The method of any one of the above aspects, wherein the compound is administered orally or parenterally.
12. The method of any one of the above aspects, wherein the compound is administered through at least one route selected from the group consisting of orally, dermally, subcutaneously, intravenously, intraarterially, intramuscularly, intraperitoneally, transmucosally, via inhalation, transnasally, ophthalmically, via an inner ear and vaginally.
13. The method of any one of the above aspects, wherein the compound is administered in combination with at least one material selected from the group consisting of a neuraminidase inhibitor, an RNA-dependent RNA polymerase inhibitor, an M2 protein inhibitor, a PB2 Cap binding inhibitor, a HA maturation inhibitor, a recombinant sialidase, a re-assemble inhibitor, RNA interference compound, a receptor of hemagglutinin binding inhibitor, a membrane of HA fusion inhibitor, a NP nuclear translocation inhibitor, a CXCR inhibitor, a CRM1 inhibitor, an anti-HA antibody and an immunological agent.
14. The method of any one of the above aspects, wherein the compound is administered in combination with at least one compound selected from the group consisting of oseltamivir, zanamivir, peramivir, laninamivir, favipiravir, amantazine, flumazine, VX-787, MHAA4549A, TCN-032, VIS-410, CR-8020, CR-6261, CT-P27 and MEDI-8852.
15. The method of any one of the above aspects, wherein the onset of influenza in the subject is when the subject has a virus titer sufficient to cause a symptom of influenza to be exhibited in the subject, wherein the onset of influenza is at least one of (1) when a body temperature of the subject increases from a normal temperature of the subject; and (2) when the subject exhibits at least one of a systemic symptom and a respiratory symptom.
16. The method of any one of the above aspects, wherein the systemic symptom includes at least one of headache, feverishness, chills, muscular pain, joint pain, and fatigue.
17. The method of any one of the above aspects, wherein the respiratory symptom includes at least one selected from the group consisting of coughing, sore throat, and nasal congestion.
18. The method of any one of the above aspects, wherein the effective amount is sufficient to alleviate a symptom of influenza in the subject as compared to a symptom that the subject has when the compound is first administered to the subject.
19. The method of any one of the above aspects, wherein the effective amount is sufficient to reduce an amount of the virus in the subject as compared to an amount of the virus that the subject has when the compound is first administered to the subject.
20. The method of any one of the above aspects, wherein the compound is administered when a virus titer is at least 0.7 log₁₀ TCID₅₀/mL.
21. The method of any one of the above aspects, wherein the compound is administered at least about 48 hours after and on or before about 120 hours after the onset of influenza in the subject.
22. The method of any one of the above aspects, wherein the compound is administered in at least one form selected from the group consisting of a tablet, powder, a granule, a capsule, a pill, a film, a suspension, an emulsion, an elixir, a syrup, lemonade, spirit, aromatic water, extract, decoction and tincture.
23. The method of any one of the above aspects, wherein the compound is administered in at least one form selected from the group consisting of a sugar-coated tablet, a film-coated tablet, an enteric-coated tablet, a sustained-release tablet, a troche tablet, a sublingual tablet, a buccal tablet, a chewable tablet, an orally disintegrated tablet, a dry syrup, a soft capsule, a micro capsule or a sustained-release capsule.
24. The method of any one of the above aspects, wherein the compound is administered in at least one form selected from the group consisting of an injection, an infusion, an eye drop, a nose drop, an ear drop, an aerosol, an inhalation, a lotion, an impregnation, a liniment, a mouthwash, an enema, an ointment, a plaster, a jelly, a cream, a patch, a cataplasm, an external powder or a suppository.
25. A method for treating influenza, comprising: reading a dosage instruction on a package insert or in a package for a pharmaceutical formulation comprising a compound having one of the following formulae:

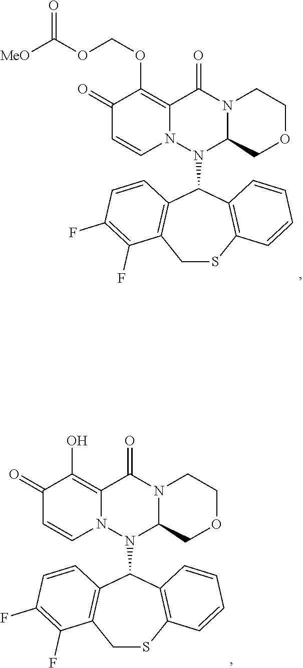 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or its pharmaceutically salt thereof; and administering initially the pharmaceutical formulation at least about 48 hours after an onset of influenza in the subject, in accordance with the dosage instruction.
26. A use of a compound having one of the following formulae:

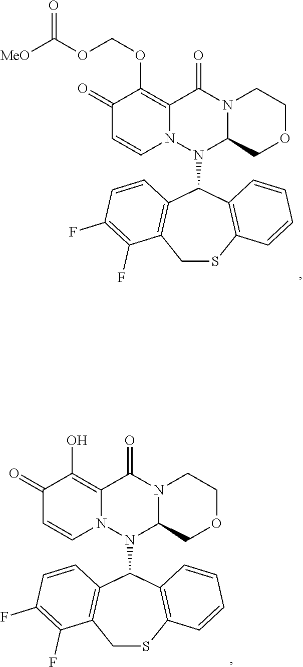 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or its pharmaceutically acceptable salt thereof, for preparation of a medicament for treating a subject having an influenza virus, wherein the treatment includes administering an effective amount of the compound to the subject having an influenza virus, and wherein the compound is administered initially at least about 48 hours after an onset of influenza in the subject.
27. A pharmaceutical composition useful for treating a subject having an influenza virus, wherein the treatment comprises administering an effective amount of a compound to the subject having an influenza virus, wherein the compound is administered initially at least about 48 hours after an onset of influenza in the subject, and wherein the pharmaceutical composition comprises the compound, which is a compound having one of the following formulae:

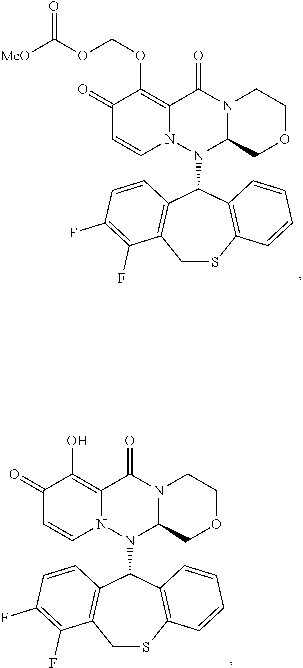 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or its pharmaceutically acceptable salt thereof.

While the disclosed compounds and methods have been described in conjunction with a preferred embodiment, it will be apparent to one skilled in the art that other objects and refinements of the disclosed compounds and methods may be made within the purview and scope of the disclosure.

The disclosure, in its various aspects and disclosed forms, is well adapted to the attainment of the stated objects and advantages of others. The disclosed details are not to be taken as limitations on the claims.

28. A package, comprising a pharmaceutical formulation comprising a compound having one of the following formulae:

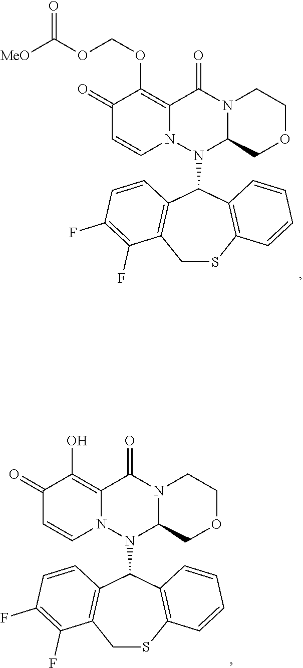 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]

or its pharmaceutically salt thereof; and a dosage instruction on a package insert or in a package for administering initially the pharmaceutical formulation at least about 48 hours after an onset of influenza in a subject.
29. The method of any one of the above aspects, wherein the subject is a patient not not a patient who requires hospitalization for severe influenza or who requires an extension of hospitalization because of influenza infection during the hospitalization.
30. The method of any one of the above aspects, wherein the subject is not a patient who has severity and complication risk factors or who requires hospitalization for severe influenza or who requires an extension of hospitalization because of influenza infection during the hospitalization.

What is claimed is:

1. A method for treating influenza, comprising:
administering an effective amount of a compound to a subject having an influenza virus,
wherein the compound is administered initially at least about 48 hours after an onset of influenza in the subject, and
wherein the compound has one of the following formulae: 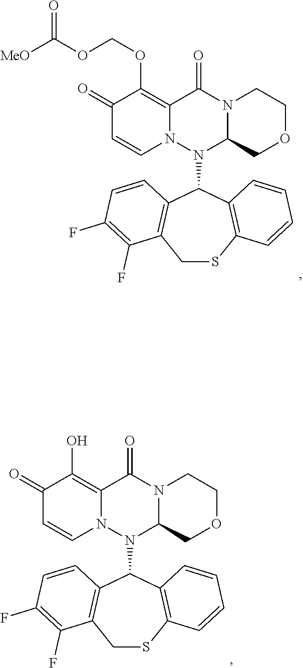 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]
or a pharmaceutically acceptable salt thereof.

2. The method of claim 1, wherein the effective amount of the compound is in a range from about 0.1 to about 240 mg.

3. The method of claim 1, wherein the effective amount of the compound is in a range from about 5 to about 80 mg.

4. The method of claim 1, wherein the effective amount of the compound is in a range from about 40 to about 80 mg.

5. The method of claim 1, wherein the effective amount of the compound is in a range from about 10 to about 80 mg per dose.

6. The method of claim 1, wherein the compound is administered only one time.

7. The method of claim 1, wherein the compound is administered only one time, two times or three times.

8. The method of claim 1, wherein the compound is administered orally or parenterally.

9. The method of claim 1, wherein the compound is administered through at least one route selected from the group consisting of orally, dermally, subcutaneously, intravenously, intraarterially, intramuscularly, intraperitoneally, transmucosally, via inhalation, transnasally, ophthalmically, via an inner ear and vaginally.

10. The method of claim 1, wherein the compound is administered in combination with at least one material selected from the group consisting of a neuraminidase inhibitor, an RNA-dependent RNA polymerase inhibitor, an M2 protein inhibitor, a PB2 Cap binding inhibitor, a HA maturation inhibitor, a recombinant sialidase, a re-assemble inhibitor, RNA interference compound, a receptor of hemagglutinin binding inhibitor, a membrane of HA fusion inhibitor, a NP nuclear translocation inhibitor, a CXCR inhibitor, a CRM1 inhibitor, an anti-HA antibody and an immunological agent.

11. The method of claim 1, wherein the compound is administered in combination with at least one compound selected from the group consisting of oseltamivir, zanamivir, peramivir, laninamivir, favipiravir, amantazine, flumazine, VX-787, MHAA4549A, TCN-032, VIS-410, CR-8020, CR-6261, CT-P27 and MEDI-8852.

12. The method of claim 1, wherein the onset of influenza in the subject is when the subject has a virus titer sufficient to cause a symptom of influenza to be exhibited in the subject, wherein the onset of influenza is at least one of (1) when a body temperature of the subject increases from a normal temperature of the subject; and (2) when the subject exhibits at least one of a systemic symptom and a respiratory symptom.

13. The method of claim 12, wherein the systemic symptom includes at least one of headache, feverishness, chills, muscular pain, joint pain, and fatigue.

14. The method of claim 12, wherein the respiratory symptom includes at least one selected from the group consisting of coughing, sore throat, and nasal congestion.

15. The method of claim 1, wherein the effective amount is sufficient to alleviate a symptom of influenza in the subject as compared to a symptom that the subject has when the compound is first administered to the subject.

16. The method of claim 1, wherein the effective amount is sufficient to reduce an amount of the virus in the subject as compared to an amount of the virus that the subject has when the compound is first administered to the subject.

17. The method of claim 1, wherein the compound is administered when a virus titer is at least 0.7 log₁₀ TCID₅₀/mL.

18. The method of claim 1, wherein the compound is administered at least about 48 hours after and on or before about 120 hours after the onset of influenza in the subject.

19. The method of claim 1, wherein the compound is administered in at least one form selected from the group consisting of a tablet, powder, a granule, a capsule, a pill, a film, a suspension, an emulsion, an elixir, a syrup, lemonade, spirit, aromatic water, extract, decoction and tincture.

20. The method of claim 1, wherein the compound is administered in at least one form selected from the group consisting of a sugar-coated tablet, a film-coated tablet, an enteric-coated tablet, a sustained-release tablet, a troche tablet, a sublingual tablet, a buccal tablet, a chewable tablet, an orally disintegrated tablet, a dry syrup, a soft capsule, a micro capsule or a sustained-release capsule.

21. The method of claim 1, wherein the compound is administered in at least one form selected from the group consisting of an injection, an infusion, an eye drop, a nose drop, an ear drop, an aerosol, an inhalation, a lotion, an impregnation, a liniment, a mouthwash, an enema, an ointment, a plaster, a jelly, a cream, a patch, a cataplasm, an external powder or a suppository.

22. A method for treating influenza, comprising: reading a dosage instruction on a package insert or in a package for a pharmaceutical formulation comprising a compound having one of the following formulae: 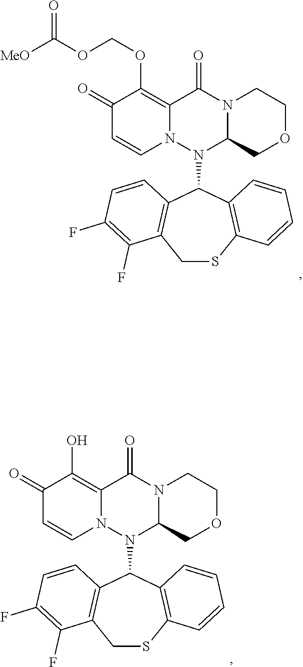 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]
or a pharmaceutically salt thereof; and administering initially the pharmaceutical formulation at least about 48 hours after an onset of influenza in the subject, in accordance with the dosage instruction.

23. A use of a compound having one of the following formulae: 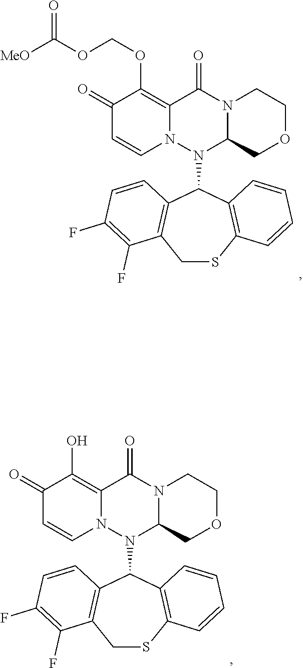 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]
or a pharmaceutically acceptable salt thereof, for preparation of a medicament for treating a subject having an influenza virus, wherein the treatment includes administering an effective amount of the compound to the subject having an influenza virus, and wherein the compound is administered initially at least about 48 hours after an onset of influenza in the subject.

24. A pharmaceutical composition useful for treating a subject having an influenza virus, wherein the treatment comprises administering an effective amount of a compound to the subject having an influenza virus, wherein the compound is administered initially at least about 48 hours after an onset of influenza in the subject, and wherein the pharmaceutical composition comprises the compound, which is a compound having one of the following formulae: 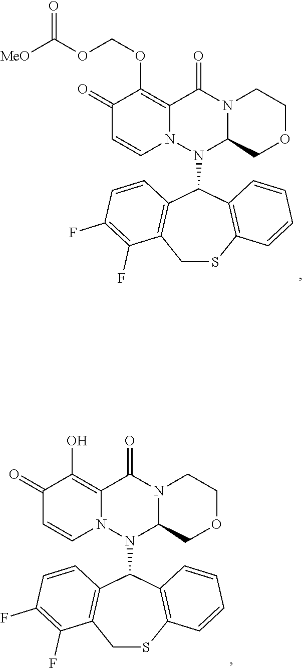 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]
or a pharmaceutically acceptable salt thereof.

25. A package, comprising a pharmaceutical formulation comprising a compound having one of the following formulae: 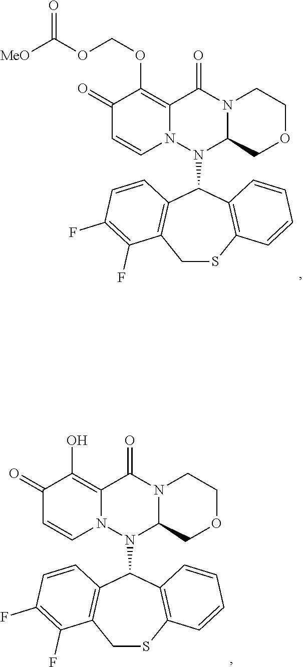 [SMILES: COC(=O)OCOc1c2n(ccc1=O)N([C@@H]1c3ccccc3SCc3c1ccc(F)c3F)[C@@H]1COCCN1C2=O ; O=C1c2c(O)c(=O)ccn2N([C@@H]2c3ccccc3SCc3c2ccc(F)c3F)[C@@H]2COCCN12]
or a pharmaceutically salt thereof; and a dosage instruction on a package insert or in a package for administering initially the pharmaceutical formulation at least about 48 hours after an onset of influenza in a subject.

26. The method of claim 1, wherein the subject is not a patient who requires hospitalization for severe influenza or who requires an extension of hospitalization because of influenza infection during the hospitalization.

27. The method of claim 1, wherein the subject is not a patient who has severity and complication risk factors or who requires hospitalization for severe influenza or who requires an extension of hospitalization because of influenza infection during the hospitalization.
